SEQUENCE LISTING (12 sequences)

SEQ ID NO: 1 - protein, 28 aa, Mus musculus
GSSFLSPEHQKAQQRKESKKPPAKLQPR

SEQ ID NO: 2 - protein, 28 aa, Homo sapiens
GSSFLSPEHQRVQQRKESKKPPAKLQPR

SEQ ID NO: 3 - protein, 28 aa, Macaca mulatta
GSSFLSPEHQRAQQRKESKKPPAKLQPR

SEQ ID NO: 4 - protein, 28 aa, Meriones unguiculatus
GSSFLSPEHQKTQQRKESKKPPAKLQPR

SEQ ID NO: 5 - protein, 28 aa, Rattus norvegicus
GSSFLSPEHQKAQQRKESKKPPAKLQPR

SEQ ID NO: 6 - protein, 28 aa, Canis canis
GSSFLSPEHQKLQQRKESKKPPAKLQPR

SEQ ID NO: 7 - protein, 28 aa, Sus scrofa
GSSFLSPEHQKVQQRKESKKPAAKLKPR

SEQ ID NO: 8 - protein, 27 aa, Ovis aries
GSSFLSPEHQKLQRKEPKKPSGRLKPR

SEQ ID NO: 9 - protein, 27 aa, Bos taurus
GSSFLSPEHQKLQRKEAKKPSGRLKPR

SEQ ID NO: 10 - protein, 28 aa, Artificial Sequence
  active (octanoylated) ghrelin
  MOD_RES 3: octanol carboxylic acid
GSSFLSPEHQKAQQRKESKKPPAKLQPR

SEQ ID NO: 11 - protein, 5 aa, Artificial Sequence
  GOAT inhibitor
  MOD_RES 3: octanoyl acyl group
  MOD_RES 5: CONH2 group
GSSFL

SEQ ID NO: 12 - protein, 21 aa, Artificial Sequence
  GOAT inhibitor
  MOD_RES 3: octanoyl-CoA acyl group
  MOD_RES 10: fusion peptide 6-aminohexanoic acid
GSSFLSPEHQYGRKKRRQRRR

(19) United States
(12) Patent Application Publication
(10) Pub. No.: US 2017/0275249 A1
(43) Pub. Date: Sep. 28, 2017

(54) SMALL LIPOPEPTIDOMIMETIC INHIBITORS OF GHRELIN O-ACYL TRANSFERASE

(71) Applicant: THE REGENTS OF THE UNIVERSITY OF CALIFORNIA, Oakland, CA (US)

(72) Inventors: Patrick G. Harran, Santa Monica, CA (US); Ryan A. Hollibaugh, Los Angeles, CA (US); Haixia Liu, Pudong (CN)

(21) Appl. No.: 15/512,365

(22) PCT Filed: Sep. 16, 2015
(86) PCT No.: PCT/US15/50499

§ 371 Date: Mar. 17, 2017

Related U.S. Application Data
(60) Provisional application No. 62/051,701, filed on Sep. 17, 2014.

(51) Int. Cl. C07D 207/06; C07D 207/09; C07D 487/08; C07D 403/12; C07D 403/06; C07D 471/04

(52) U.S. Cl. CPC C07D 207/06; C07D 403/06; C07D 471/04; C07D 487/08; C07D 403/12; C07D 207/09

(57) ABSTRACT

Compositions and methods are disclosed that relate to small molecule lipopeptidomimetic inhibitors of mammalian ghrelin O-acyl transferase (GOAT). Compounds of general Formula (I) and substructures thereof, i.e., Formulae (II), (IIa), (IIa1), (IIa2), (IIb), (IIb1), (IIb2), (IIc) and (III), are shown to exhibit potent inhibition of the octanoylation of ghrelin peptide, where the resulting non-octanoylated (des-acyl) form of ghrelin lacks GHSr ligand activity that is associated with weight gain and insulin resistance. These and related embodiments will find uses for treating subjects known to have, or suspected of being at risk for having, a condition that would benefit from a decreased level of acylated ghrelin peptide, such as type II diabetes, impaired glucose tolerance, insulin resistance, Prader-Willi syndrome (PWS) and obesity.

### CROSS REFERENCE TO RELATED APPLICATION

This application claims the benefit under 35 U.S.C. §119(e) to U.S. Provisional Application No. 62/051,701 filed Sep. 17, 2014, which application is hereby incorporated by reference in its entirety.

### STATEMENT OF GOVERNMENT INTEREST

This invention was made with government support under Grant Nos. T32 GM 8496-18, awarded by the National Institutes of Health. The U.S. Government may have certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 720156_405WO_SEQUENCE_LISTING.txt. The text file is 3.9 KB, was created on Sep. 16, 2015, and is being submitted electronically via EFS-Web.

### BACKGROUND

Technical Field

The presently disclosed invention embodiments relate to compositions and methods for treating type II diabetes, Prader-Willi syndrome, obesity, and related metabolic conditions and disorders. In particular, disclosed herein is a class of small molecules that inhibit formation of the active form of ghrelin, an acylated peptide hormone that may increase appetite and fat retention and that decreases glucose tolerance.

Description of the Related Art

Type II diabetes is one of the most prevalent obesity-related diseases. There are estimated to be 29 million people in the U.S. who have type II diabetes, and over 86 million pre-diabetics who have a high likelihood of progressing from a pre-diabetic status (e.g., impaired glucose tolerance, insulin resistance) to type II diabetes (Centers for Disease Control and Prevention, National Diabetes Statistics Report, 2014. http://www.cdc.gov/diabetes/pubs/statsreport14.htm). Impaired glucose tolerance is defined as an abnormally high (between 140 and 200 mg/dL) plasma glucose concentration two hours after a 75 g oral glucose tolerance test. (Nathan et al., 2007 Diabetes Care 30:753-759). Insulin resistance is defined as “a subnormal biologic response to a given concentration of insulin.” (Moller, D. E. and Flier, J. S. 1991 New England Journal of Medicine 325:938-948; see also, e.g., Reaven, 2005 Annu. Rev. Nutr. 25:391.) The National Institute of Diabetes and Digestive and Kidney Diseases (NIDDK, Bethesda, Md.) has selected a set of criteria used for diagnosing insulin resistance syndrome. These criteria include large waist size (waist measurement of 40 inches or more for men and 35 inches or more for women), high triglycerides in the blood (triglyceride level of 150 milligrams per deciliter (mg/dL) or above, or taking medication for elevated triglyceride level), abnormal levels of cholesterol in the blood (HDL, or good, cholesterol level below 40 mg/dL for men and below 50 mg/dL for women, or taking medication for low HDL), high blood pressure (blood pressure level of 130/85 or above, or taking medication for elevated blood pressure), and higher than normal blood glucose levels (fasting blood glucose level of 100 mg/dL or above, or taking medication for elevated blood glucose). (Alberti et al., Circulation. 2009; 120:1640-1645.) (See, e.g., http://diabetes.niddk.nih.gov/dm/pubs/insulinresistance/#metabolic).

Medical costs for the treatment of diabetes were estimated by the American Diabetes Association to be $176 billion in 2012 alone. Despite these statistics, treatment for diabetes has remained largely unchanged. Insulin injections, coupled with careful control of food intake and blood sugar monitoring remains the most common treatment for this condition. See also: Reaven, G. M., Annual Review of Nutrition 25: 391-406.

Diabetes is a complex disease, but is essentially the inability of an individual to correctly regulate blood glucose levels. In healthy individuals, blood glucose levels rise following food intake and in response, the polypeptide hormone insulin is released from the pancreas into the bloodstream. Insulin functions, via biological signal transduction through hepatic cell surface insulin receptors, to inhibit glucose release from the liver. This system for regulating the level of glucose in the blood is known as glucose tolerance. Individuals with type II diabetes are unable to regulate their blood glucose level by this mechanism due to the development of insulin resistance, in which cells continue to release glucose even in the presence of insulin.

Prader-Willi Syndrome (PWS) is a rare genetic abnormality in humans that is characterized by partial deletion of chromosome 15q. PWS is associated with insatiable appetite leading to dramatic early-onset obesity in children. There are estimated to be approximately 400,000 PWS patients worldwide.

Obesity is a metabolic syndrome caused by a chronic energy imbalance that results in a spectrum of related conditions that represent some of the leading causes of preventable death, according to the U.S. Centers for Disease Control (CDC). These obesity-related conditions include heart disease, stroke, type II diabetes and certain types of cancer. Obesity has reached epidemic levels in contemporary society (National Center for Health Statistics Health E-Stats. Prevalence of overweight, obesity and extreme obesity among adults: United States, trends 1976-80 through 2005-2006, 2008; Flegal et al., 2010 JAMA 303:235-241; Finkelstein et al., 2009 Health Affairs 28:822-831). In the United States, an estimated 35% of Americans are obese (body mass index, BMI>30) and the obese human population worldwide was estimated at 617 million people in 2012, with an estimated 95 million people worldwide classified as severely obese (BMI>40). The estimated annual medical cost of obesity in the U.S. was $147 billion in 2008, according to the CDC.

Ghrelin is a polypeptide hormone that is produced primarily in the digestive tract. Ghrelin is an endogenous ligand for the growth hormone secretagogue receptor (GHSr), and was originally identified in the context of growth hormone release (Kojima et al., 1999 Nature 402:656-660). It subsequently became clear, however, that ghrelin plays larger roles in energy homeostasis. For instance, binding by ghrelin to GHSr in pancreatic islet cells blocked insulin secretion (Dezaki et al., 2007 Diabetes 56:2319-2327). Thus, blockade of ghrelin production may usefully restore insulin release in response to glucose challenge.

In PWS patients, ghrelin levels are increased several fold above normal levels, and it has been speculated that an agent that could prevent the action of ghrelin on its receptor could represent a mode of treatment for this condition (Horvath et al., 2003 Current Pharmaceutical Design 9:1383-1395).

Early reports indicated that administration of ghrelin to rodent or human subjects stimulated feeding behavior, and chronic ghrelin administration promoted excess adiposity (Wren et al., 2001 Diabetes 50:2540-2547; Wortley et al., 2005 J. Clin. Invest. 115:3573-3578). More recent results in rodent models have, however, called this phenotype into question. For example, transgenic mice lacking either ghrelin or its receptor ate and gained weight at the same rate as control animals. In the same study, a consistently observed phenotype following genetic knockout of ghrelin or its receptor was an increased susceptibility to hypoglycemia under conditions of severe calorie restriction (Zhao et al., 2010 Proc. Nat. Acad. Sci. USA 107:7467-7472). In another study, ghrelin doses required to promote feeding in mice or humans were significantly higher than those that were achieved physiologically (Macfarlane et al., 2014 Cell Metabolism. 20:54-60). In transgenic mice engineered to have ghrelin secreting cells that also expressed the receptor for diphtheria toxin, circulating ghrelin concentrations were diminished by >85% upon injection of diphtheria toxin. In these animals there was no significant difference in feeding behavior or weight gain between control and ghrelin ablated mice fed a normal or high fat diet (Macfarlane et al., 2014 Cell Metabolism. 20:54-60). This murine model creatively established an acute blockade of ghrelin signaling; importantly, it remains unknown whether these results will be predictive of a bona fide pharmacological inhibitor of ghrelin signaling in higher mammals and humans.

Ghrelin biosynthesis takes place principally in the gastric mucosa and involves maturation from a 117 amino-acid precursor polypeptide that undergoes two proteolytic processing steps and one acylation to yield a 28-residue peptide that has been octanoylated on the serine residue at peptide position three. Romero et al., 2010 Eur. Jl. of Endocrinol. 163:1-8. Octanoylation of ghrelin is required for ghrelin's observed biological activity as a GHSr ligand; the non-acylated form of the ghrelin polypeptide (des-acyl ghrelin) lacks such activity. After being produced in the gastric mucosa, both ghrelin and des-acyl ghrelin are exported to the bloodstream.

The enzyme that catalyzes the octanoylation of ghrelin in gastric mucosa was discovered in 2008 and termed ghrelin O-acyltransferase (GOAT). GOAT uses octanoyl CoA as a donor for the acylation reaction. (FIG. 1; Yang et al., 2008 Cell 132:387-396; Gutierrez et al., 2008 Proc. Nat. Acad. Sci. USA 105:6320:6325) To date ghrelin is the only known biological substrate for GOAT octanoylation. Inhibition of GOAT results in a decrease in circulating levels of active ghrelin, which decrease is believed usefully to be reflected in, e.g., reduced blockade of insulin secretion and/or potentially reduced stimulation of food intake and/or reduced adiposity.

As discussed above, the action of ghrelin on pancreatic islet beta-cells directly blocks glucose dependent increases in intracellular calcium and the associated efflux of insulin. GOAT inhibitors are thus believed to contribute to the restoration of an insulin response to glucose challenge, and may have the potential to treat diabetes. Preliminary in vivo studies using a cell permeable, bi-substrate GOAT inhibitor support this view. Barnett et al., 2010 Science 330:1689-1692.

Additionally, because ghrelin is the only known biological substrate of GOAT octanoylation activity and GOAT is the only enzyme known to be capable of octanoylating ghrelin (Yang et al., 2008 Cell 132:387-396), inhibitors of GOAT have potential to be selective for decreasing the amount of active ghrelin while having minimal “on-target” side effects. Moreover, given that both ghrelin and GOAT are produced primarily in the digestive tract (Kojima et al., 1999 Nature 402:656-660; Yang et al., 2008 Cell 132:387-396), effective inhibitors need not access the CNS (i.e., need not be capable of crossing the blood-brain barrier).

A number of GOAT inhibitors have been described. Yang et al. (2008 Proc. Natl. Acad. Sci. USA 105:10750) identified an octanoylated pentapeptide derived from the ghrelin N-terminus that was effective as a GOAT inhibitor in vitro, but which did not inhibit GOAT in cultured cells. Garner et al. (2011 Chem. Commun. 47:7512-7514) described a 2-napthylglycine derivative that inhibited GOAT when tested using an enzyme-linked immunosorbent (ELISA) based assay. Barnett et al. (2010 Science 330:1689-1692) generated a fusion peptide conjugate derived from ghrelin, octanoyl-CoA, and a Tat peptide sequence to promote cellular uptake, which conjugate exhibited GOAT-inhibitory activity in vitro as well as in vivo. In a mouse model, the fusion peptide conjugate effected a reduction in weight gain and improved response to glucose stress. U.S. Pat. Nos. 8,329,745 and 8,013,015 disclose a class of vinylglycine-derived GOAT inhibitors. JP2013/055605 and WO2013/125732 disclose polysubstituted benzothiophenes as GOAT inhibitors. Despite the emergence of these GOAT inhibitors, challenges remain with respect to efficacy, potency, substrate accessibility, pharmacokinetic properties, and other criteria important to suitability for development as a therapeutic.

Clearly there remains a need in the art for improved GOAT inhibitors that are amenable to development as effective agents for treating type II diabetes, obesity, Prader-Willi Syndrome and related metabolic conditions and disorders. The presently described compositions and methods address this need and offer other related advantages.

### BRIEF SUMMARY

Provided herein are compounds and pharmaceutical compositions that inhibit ghrelin O-acyltransferase (GOAT) catalytic activity specifically and potently, including inhibiting GOAT activity in cells with little or no detectable general cellular cytotoxicity, and method of using the same.

One embodiment provides a compound represented by Formula (I):

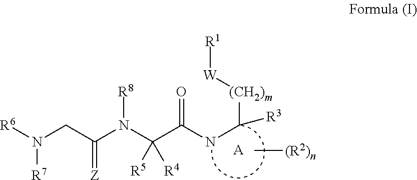

wherein:

A is an N-heterocyclic ring;

W is —C(G)-Y—, —NR⁹—, —O—, —C(G)-, —CH═CH—, or —C≡C—;

G is O or S;

Y is NR⁹ or O;

Z is O, S or NR⁹;

m is 0, 1 or 2;

n is 0, 1, 2, 3 or 4;

R¹ is alkyl, alkenyl, alkynyl, haloalkyl, aryl, aralkyl, heterocyclyl, heterocyclylalkyl, heteroaryl or heteroarylalkyl;

each R² is independently alkyl, aryl, halo, hydroxyl or alkoxy; or

two adjacent R²s together with the atoms to which they are attached form a 5-, 6- or 7-member heterocyclic or carbocyclic ring;

R³ is hydrogen, alkyl, alkenyl or alkynyl;

R⁴ and R⁵ are independently hydrogen, alkyl, aralkyl, alkenyl, alkynyl, aralkenyl, cycloalkyl, cycloalkylalkyl, or haloalkyl; or

R⁴ and R² together with the atoms to which they are attached form an N-heterocyclic ring;

R⁶ and R⁷ are independently hydrogen, alkyl, alkenyl, alkyloxycarbonyl, or alkenyloxycarbonyl; or

R⁶ and R¹ are linked together to form alkylene, alkenylene or alkynylene; or

R⁶ and R⁴ are linked together to form alkylene, alkenylene or alkynylene; or

R⁸ is hydrogen or alkyl; and

R⁹ is hydrogen or alkyl,

a stereoisomer, enantiomer or tautomer thereof, a pharmaceutically acceptable salt thereof, or a prodrug thereof.

A further embodiment provides a compound represented by Formula (II):

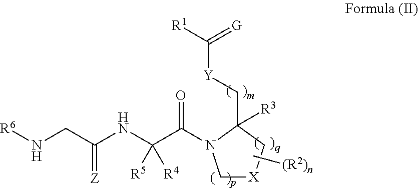

wherein:

p is 0, 1 or 2;

q is 0, 1 or 2;

m is 0, 1 or 2;

n is 0, 1, 2, 3 or 4;

X is —C(R¹⁰)₂—; —O—; —N(R¹⁰)—; or —S—;

G is O or S;

Y is NH or O;

Z is O, S or NR⁹;

R¹ is alkyl, alkenyl, alkynyl, haloalkyl, aryl, aralkyl, heterocyclyl, heterocyclylalkyl, heteroaryl or heteroarylalkyl;

each R² is independently alkyl, aryl, halo, hydroxyl or alkoxy; or

two adjacent R²s together with the atoms to which they are attached form a 5-, 6- or 7-member heterocyclic or carbocyclic ring;

R³ is hydrogen, alkyl, alkenyl or alkynyl;

R⁴ and R⁵ are independently hydrogen, alkyl, aralkyl, alkenyl, alkynyl, aralkenyl, cycloalkyl, cycloalkylalkyl, or haloalkyl;

R⁴ and R² together with the atoms to which they are attached form an N-heterocyclic ring;

R⁶ is hydrogen, alkyl, alkenyl, alkyloxycarbonyl, or alkenyloxycarbonyl; or

R⁶ and R¹ are linked together to form alkylene, alkenylene or alkynylene; or

R⁶ and R⁴ are linked together to form alkylene, alkenylene or alkynylene;

R⁹ is hydrogen or alkyl, and

each R¹⁰ is independently hydrogen, alkyl, halo, hydroxyl or alkoxy; or

R¹⁰ forms a direct bond to an adjacent atom.

Yet another embodiment provides a compound represented by Formula (IIa):

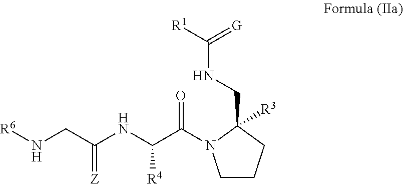

wherein:

G is O or S;

Z is O, S or NH;

R¹ is alkyl or heterocyclyl;

R³ is hydrogen, alkyl, alkenyl or alkynyl;

R⁴ is alkyl or cycloalkyl; and

R⁶ is hydrogen or alkyl.

Yet another embodiment provides a compound represented by Formula (IIa1)

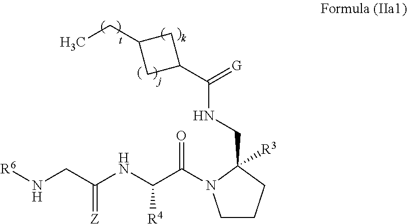

wherein:

k and j are independently 1, 2 or 3;

t is any integer between 0 to 8;

G is O or S;

Z is O, S or NH;

R³ is hydrogen, alkyl, alkenyl or alkynyl;

R⁴ is alkyl or cycloalkyl; and

R⁶ is hydrogen or alkyl.

A further embodiment provides a compound represented by Formula (IIa2):

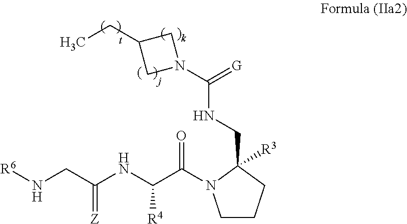

wherein:

k and j are independently 1, 2 or 3;

t is any integer between 0 to 8;

G is O or S;

Z is O, S or NH;

R³ is hydrogen, alkyl, alkenyl or alkynyl;

R⁴ is alkyl or cycloalkyl; and

R⁶ is hydrogen or alkyl.

A further embodiment provides a compound represented by Formula (IIb):

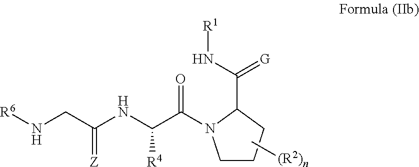

wherein:

n is 0, 1, 2;

G is O or S;

Z is O, S or NH;

R¹ is alkyl or heterocyclyl;

each R² is independently alkyl, aryl, halo, hydroxyl or alkoxy; or

two adjacent R²s together with the atoms to which they are attached form a 5-, 6- or 7-member heterocyclic or carbocyclic ring;

R⁴ is alkyl or cycloalkyl;

R⁶ is hydrogen or alkyl.

A further embodiment provides a compound represented by Formula (IIb1)

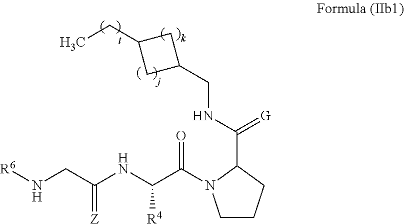

wherein:

k and j are independently 1, 2 or 3;

t is any integer between 0 to 8;

G is O or S;

Z is O, S or NH;

R⁴ is alkyl or cycloalkyl; and

R⁶ is hydrogen or alkyl.

Yet another embodiment provides a compound represented by Formula (IIb2)

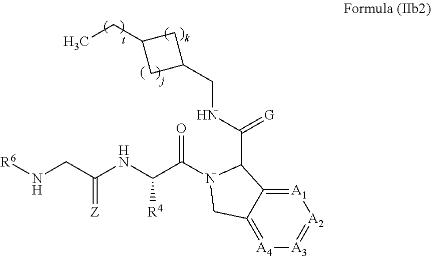

wherein:

k and j are independently 1, 2 or 3;

t is any integer between 0 to 8;

G is O or S;

Z is O, S or NH;

R⁴ is alkyl or cycloalkyl;

R⁶ is hydrogen or alkyl; and

A1, A2, A3 and A4 is independently —CH—; —N— or nothing.

A further embodiment provides a compound represented by Formula (IIc):

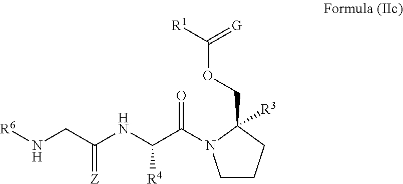

wherein:

G is O or S;

Z is O, S or NH;

R¹ is alkyl or heterocyclyl;

R³ is hydrogen, alkyl, alkenyl or alkynyl;

R⁴ is alkyl or cycloalkyl; and

R⁶ is hydrogen or alkyl.

Yet another embodiment provides a compound represented by Formula (III)

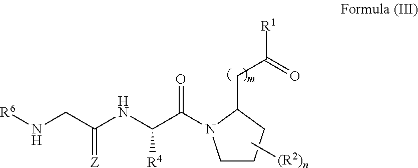

wherein:

n is 0, 1 or 2;

m is 0, 1 or 2;

Z is O, S or NH;

R¹ is alkyl or heterocyclyl;

each R² is independently alkyl, halo, hydroxyl or alkoxy; or

two adjacent R²s together with the atoms to which they are attached form a 5-, 6- or 7-member heterocyclic or carbocyclic ring;

R⁴ is alkyl or cycloalkyl; and

R⁶ is hydrogen or alkyl.

Yet another embodiment provides a pharmaceutical composition comprising a compound of any one of Formula (I) and substructures thereof, i.e., Formulae (II), (IIa), (IIa1), (IIa2), (IIb), (IIb1), (IIb2), (IIc) and (III), and a pharmaceutically acceptable excipient.

A further embodiment provides a method for substantially impairing acylation of a ghrelin peptide by a ghrelin O-acyl transferase (GOAT) enzyme, comprising contacting the GOAT enzyme with an effective amount of the compound of any one of Formula (I) and substructures thereof, i.e., Formulae (II), (IIa), (IIa1), (IIa2), (IIb), (IIb1), (IIb2), (IIc) and (III), or the pharmaceutical composition thereof.

A further embodiment provides a method for treating a subject known to have, or suspected of being at risk for having a condition that would benefit from a decreased level of acylated ghrelin peptide, comprising administering to the subject a therapeutically effective amount of the compound of any one of Formula (I) and substructures thereof, i.e., Formulae (II), (IIa), (IIa1), (IIa2), (IIb), (IIb1), (IIb2), (IIc) and (III), or the pharmaceutical composition thereof.

In various embodiments, the condition that would benefit from a decreased level of acylated ghrelin peptide comprises type II diabetes.

In other embodiments, the condition that would benefit from a decreased level of acylated ghrelin peptide comprises one or more of impaired glucose tolerance, insulin resistance, type II diabetes, Prader-Willi syndrome (PWS) and obesity.

In various embodiments, the subject is a human, a mammal, non-human primate, a mouse, a rat, a rabbit, a dog, a cat, a hamster, a gerbil, a guinea pig, a goat, a sheep, a bovine, a swine and a horse.

### BRIEF DESCRIPTION OF THE SEVERAL VIEWS OF THE DRAWINGS

FIG. 1 shows GOAT catalyzed octanoylation of des-acyl ghrelin (SEQ ID NO: 1) to form active (octanoylated) ghrelin (SEQ ID NO: 10).

FIG. 2(A) shows structures of exemplary GOAT inhibitor and negative control compound and FIG. 2(B) shows in vitro inhibitory activity of a GOAT inhibitor according to one embodiment, as compared to negative control, in a ghrelin radio-octanoylation assay performed according to Yang et al. (2008 Proc. Nat. Acad. Sci. USA 105:10750).

FIG. 3 shows an in vivo time course of serum ghrelin levels (ratio of acyl-ghrelin to total ghrelin peptide) and an exemplary GOAT inhibitor (1) concentration according to a disclosed embodiment. Wild-type mice received 80 mg/kg of (1) intraperitoneally at time zero.

FIG. 4 shows the effects on acyl ghrelin levels in GOAT/preproghrelin-expressing INS-1 cells in vitro, of varying concentrations of an exemplary compound (1) according to an embodiment and of a negative control (2).

FIG. 5 shows prior art GOAT inhibitors (SEQ ID NO: 11) and (SEQ ID NO: 12).

FIG. 6 shows comparative results of in vitro inhibitor effects of exemplary compound (1) according to an embodiment as compared to prior art GOAT inhibitors (3-6) in the ghrelin radio-octanoylation assay performed according to Yang et al. (2008 Proc. Nat. Acad. Sci. USA 105:10750).

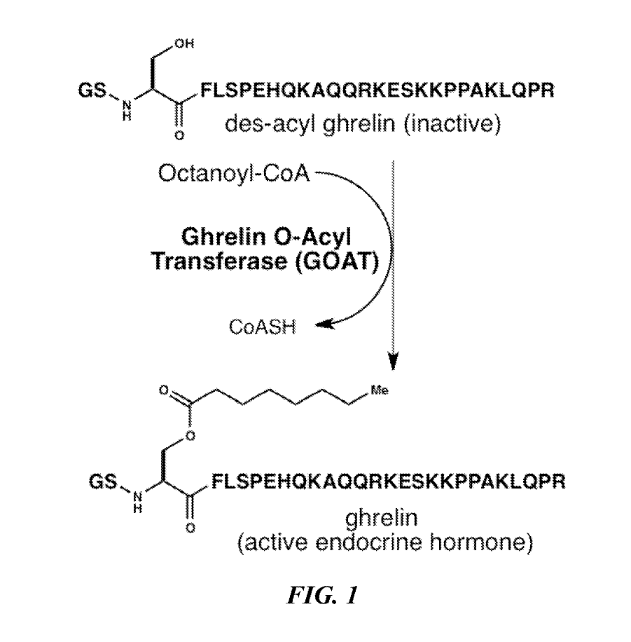

### BRIEF DESCRIPTION OF THE SEQUENCES

SEQ ID NO:1 shows the 28-amino acid murine des-acyl ghrelin peptide:

[SEQ ID NO: 1]
GSSFLSPEHQKAQQRKESKKPPAKLQPR

SEQ ID NO:2 shows the human des-acyl ghrelin peptide:

[SEQ ID NO: 2]
GSSFLSPEHQRVQQRKESKKPPAKLQPR

SEQ ID NO:3 shows the rhesus monkey des-acyl ghrelin peptide:

[SEQ ID NO: 3]
GSSFLSPEHQRAQQRKESKKPPAKLQPR

SEQ ID NO:4 shows the Mongolian gerbil des-acyl ghrelin peptide:

[SEQ ID NO: 4]
GSSFLSPEHQKTQQRKESKKPPAKLQPR

SEQ ID NO:5 shows the rat des-acyl ghrelin peptide:

[SEQ ID NO: 5]
GSSFLSPEHQKAQQRKESKKPPAKLQPR

SEQ ID NO:6 show the canine des-acyl ghrelin peptide:

[SEQ ID NO: 6]
GSSFLSPEHQKLQQRKESKKPPAKLQPR

SEQ ID NO:7 shows the porcine des-acyl ghrelin peptide:

[SEQ ID NO: 7]
GSSFLSPEHQKVQQRKESKKPAAKLKPR

SEQ ID NO:8 shows the sheep des-acyl ghrelin peptide:

[SEQ ID NO: 8]
GSSFLSPEHQKLQRKEPKKPSGRLKPR

SEQ ID NO:9 shows the bovine des-acyl ghrelin peptide:

[SEQ ID NO: 9]
GSSFLSPEHQKLQRKEAKKPSGRLKPR

These and other exemplary vertebrate ghrelin peptide sequences are disclosed in Kojima et al., 2005 Physiol Rev. 85:495-522.

### DETAILED DESCRIPTION

The presently disclosed invention embodiments relate to the identification of small molecule compounds that, unexpectedly, inhibit ghrelin O-acyltransferase (GOAT) catalytic activity specifically and potently, including inhibiting GOAT activity in cells with little or no detectable general cellular cytotoxicity. The presently disclosed compounds may be used as therapeutic agents and/or as lead compounds to develop targeted drugs to treat or reduce the risk or likelihood of occurrence of a condition that would benefit from a decreased (e.g., reduced in a statistically significant manner relative to an untreated control) level of acylated ghrelin peptide, for example, obesity, type II diabetes, and related metabolic conditions and disorders. Preferred embodiments thus relate to treatment of impaired glucose tolerance, insulin resistance, type II diabetes, and/or obesity, in each of which excessive ghrelin activity and/or overexpression of active (e.g., octanoylated) ghrelin may be a contributing factor. In certain preferred embodiments treatment is provided of a mammalian condition that would benefit from a decreased level of acylated ghrelin peptide, which in certain particularly preferred embodiments may be a human condition that would benefit from a decreased level of acylated ghrelin peptide.

As noted above, ghrelin is an endocrine hormone that is synthesized, processed and secreted by specialized cells in the stomach. Once in the circulation, ghrelin acts on pancreatic islets to block insulin secretion and in the pituitary to stimulate growth hormone release, promote feeding and regulate energy homeostasis. Described herein are novel compounds that are capable of potently blocking GOAT-catalyzed ghrelin synthesis in vitro and in vivo. According to non-limiting theory, because ghrelin and GOAT are produced primarily in the digestive tract, GOAT is a particularly attractive target for pharmacological intervention to regulate ghrelin activity where an effective GOAT inhibitor need not (and preferably would not) penetrate the central nervous system in order to down-regulate ghrelin action. Moreover, ghrelin is the only hormone known to be octanoylated, GOAT is the only known enzyme capable of octanoylating des-acyl ghrelin to generate active ghrelin, and ghrelin is the only known substrate of GOAT. Accordingly the specificity inherent in the ghrelin/GOAT system may according to certain herein disclosed embodiments be beneficially exploited by the presently disclosed GOAT inhibitors, which are thus believed to act with advantageously high selectivity to provide the present compositions and methods.

### GOAT Inhibitors

Disclosed herein are compounds of Formula (I), as well as substructures (II), (IIa), (IIa1), (IIa2), (IIb), (IIb1), (IIb2), (IIc) and (III) useful as GOAT inhibitors.

One embodiment provides a compound of Formula (I):

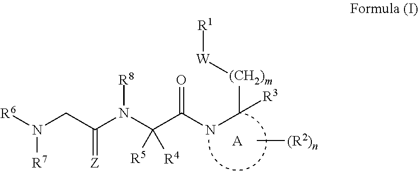

wherein:

A is an N-heterocyclic ring;

W is —C(G)-Y—, —NR⁹—, —O—, —C(G)-, —CH═CH—, or —C≡C—;

G is O or S;

Y is NR⁹ or O;

Z is O, S or NR⁹;

m is 0, 1 or 2;

n is 0, 1, 2, 3 or 4;

R¹ is alkyl, alkenyl, alkynyl, haloalkyl, aryl, aralkyl, heterocyclyl, heterocyclylalkyl, heteroaryl or heteroarylalkyl;

each R² is independently alkyl, aryl, halo, hydroxyl or alkoxy; or

two adjacent R²s together with the atoms to which they are attached form a 5-, 6- or 7-member heterocyclic or carbocyclic ring;

R³ is hydrogen, alkyl, alkenyl or alkynyl;

R⁴ and R⁵ are independently hydrogen, alkyl, aralkyl, alkenyl, alkynyl, aralkenyl, cycloalkyl, cycloalkylalkyl, or haloalkyl; or

R⁴ and R² together with the atoms to which they are attached form an N-heterocyclic ring;

R⁶ and R⁷ are independently hydrogen, alkyl, alkenyl, alkyloxycarbonyl, or alkenyloxycarbonyl; or

R⁶ and R¹ are linked together to form alkylene, alkenylene or alkynylene; or

R⁶ and R⁴ are linked together to form alkylene, alkenylene or alkynylene; or

R⁸ is hydrogen or alkyl; and

R⁹ is hydrogen or alkyl,

a stereoisomer, enantiomer or tautomer thereof, a pharmaceutically acceptable salt thereof, or a prodrug thereof.

Of the compounds of Formula (I), as set forth above in, another embodiment provides a compound, wherein:

W is —C(G)-Y—;

Y is NH or O

G is O or S; and

m is 0, 1 or 2.

Of this embodiment, one embodiment is a compound represented by Formula (II):

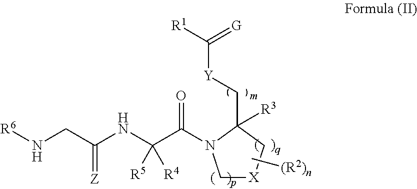

wherein:

p is 0, 1 or 2;

q is 0, 1 or 2;

m is 0, 1 or 2;

n is 0, 1, 2, 3 or 4;

X is —C(R¹⁰)₂—; —O—; —N(R¹⁰)—; or —S—;

G is O or S;

Y is NH or O;

Z is O, S or NR⁹;

R¹ is alkyl, alkenyl, alkynyl, haloalkyl, aryl, aralkyl, heterocyclyl, heterocyclylalkyl, heteroaryl or heteroarylalkyl;

each R² is independently alkyl, aryl, halo, hydroxyl or alkoxy; or

two adjacent R²s together with the atoms to which they are attached form a 5-, 6- or 7-member heterocyclic or carbocyclic ring;

R³ is hydrogen, alkyl, alkenyl or alkynyl;

R⁴ and R⁵ are independently hydrogen, alkyl, aralkyl, alkenyl, alkynyl, aralkenyl, cycloalkyl, cycloalkylalkyl, or haloalkyl;

R⁴ and R² together with the atoms to which they are attached form an N-heterocyclic ring;

R⁶ is hydrogen, alkyl, alkenyl, alkyloxycarbonyl, or alkenyloxycarbonyl; or

R⁶ and R¹ are linked together to form alkylene, alkenylene or alkynylene; or

R⁶ and R⁴ are linked together to form alkylene, alkenylene or alkynylene;

R⁹ is hydrogen or alkyl, and

each R¹⁰ is independently hydrogen, alkyl, halo, hydroxyl or alkoxy; or

R¹⁰ forms a direct bond to an adjacent atom.

Of the compounds of Formula (II), as set forth above, another embodiment is a compound represented by Formula (IIa):

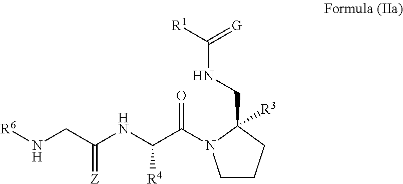

wherein:

G is O or S;

Z is O, S or NH;

R¹ is alkyl or heterocyclyl;

R³ is hydrogen, alkyl, alkenyl or alkynyl;

R⁴ is alkyl or cycloalkyl; and

R⁶ is hydrogen or alkyl.

A further embodiment of Formula (IIa) provides a compound wherein R¹ is alkyl.

Yet another embodiment of Formula (IIa) provides a compound wherein

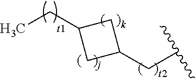

wherein,

k and j are independently 1, 2 or 3; and

t1 and t2 are independently any integer between 0 to 8.

Of the compounds of Formula (IIa), as set forth above, another embodiment is represented by Formula (IIa1)

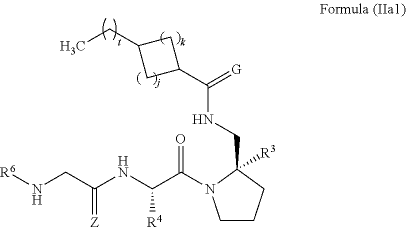

wherein:

k and j are independently 1, 2 or 3;

t is any integer between 0 to 8;

G is O or S;

Z is O, S or NH;

R³ is hydrogen, alkyl, alkenyl or alkynyl;

R⁴ is alkyl or cycloalkyl; and

R⁶ is hydrogen or alkyl.

Of the compounds of Formula (IIa), as set forth above, another embodiment is represented by Formula (IIa2):

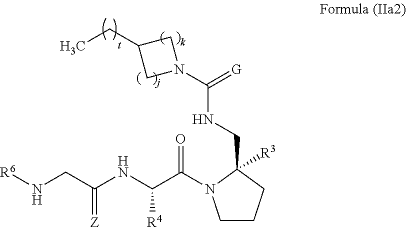

wherein:

k and j are independently 1, 2 or 3;

t is any integer between 0 to 8;

G is O or S;

Z is O, S or NH;

R³ is hydrogen, alkyl, alkenyl or alkynyl;

R⁴ is alkyl or cycloalkyl; and

R⁶ is hydrogen or alkyl.

A further embodiment of any one of Formula (IIa) and substructures Formulae (IIa1) and (IIa2) provides a compound wherein R⁴ is t-butyl.

A further embodiment of any one of Formula (IIa) and substructures Formulae (IIa1) and (IIa2) provides a compound wherein R⁴ is cyclohexyl or cyclopentyl.

A further embodiment of any one of Formula (IIa) and substructures Formulae (IIa1) and (IIa2) provides a compound wherein Z is S or O.

A further embodiment of any one of Formula (IIa) and substructures Formulae (IIa1) and (IIa2) provides a compound wherein G is O.

With reference to the following structure, Table 1 shows a number of specific embodiments of Formula (IIa) or (IIa1), accompanied by their respective inhibitory activities against GOAT:

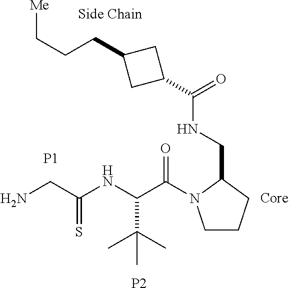

TABLE 1

| Entry | IC₅₀ | P1 | P2 | Core | Side Chain |
|---|---|---|---|---|---|
| 1 | 2000 nM | 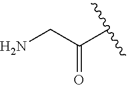 [SMILES: *C(=O)CN] | 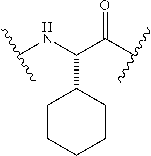 [SMILES: *N[C@H](C(*)=O)C1CCCCC1] | 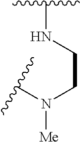 | 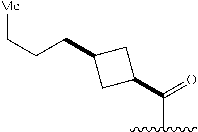 |
| 2 | >3000 nM | 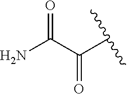 [SMILES: *C(=O)C(N)=O] | 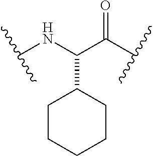 [SMILES: *N[C@H](C(*)=O)C1CCCCC1] | 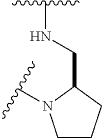 | 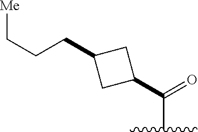 |
| 3 | 300 nM | 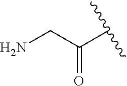 [SMILES: *C(=O)CN] | 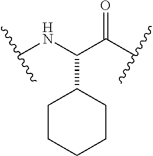 [SMILES: *N[C@H](C(*)=O)C1CCCCC1] | 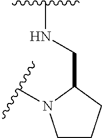 | 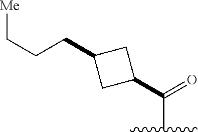 |
| 4 | 900 nM | 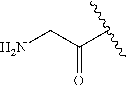 [SMILES: *C(=O)CN] | 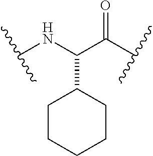 [SMILES: *N[C@H](C(*)=O)C1CCCCC1] | 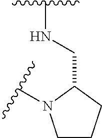 | 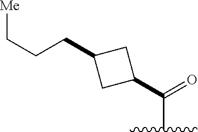 |
| 5 | >3000 nM | 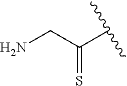 [SMILES: *C(=S)CN] | 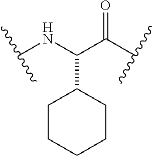 [SMILES: *N[C@H](C(*)=O)C1CCCCC1] | 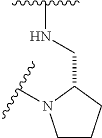 | 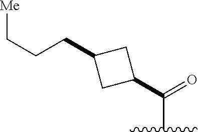 |
| 6 | 300 nM | 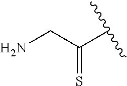 [SMILES: *C(=S)CN] | 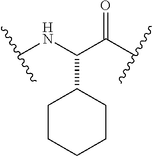 [SMILES: *N[C@H](C(*)=O)C1CCCCC1] | 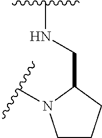 | 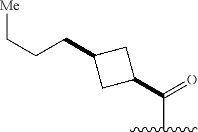 |
| 7 | >3000 nM | 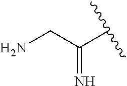 [SMILES: *C(=N)CN] | 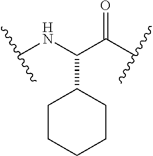 [SMILES: *N[C@H](C(*)=O)C1CCCCC1] | 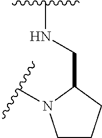 | 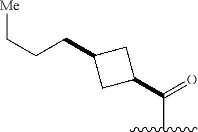 |
| 8 | 300 nM | 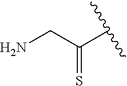 [SMILES: *C(=S)CN] | 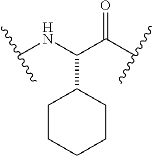 [SMILES: *N[C@H](C(*)=O)C1CCCCC1] | 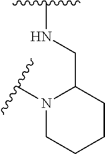 [SMILES: *NCC1CCCCN1*] | 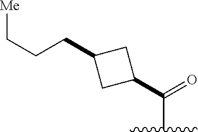 |
| 9 | >3000 nM | H | 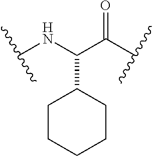 [SMILES: *N[C@H](C(*)=O)C1CCCCC1] | 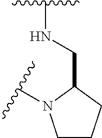 | 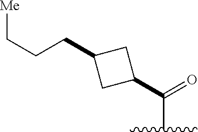 |
| 10 | 500 uM | 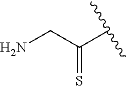 [SMILES: *C(=S)CN] | 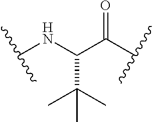 [SMILES: *N[C@H](C(*)=O)C(C)(C)C] | 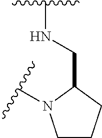 | 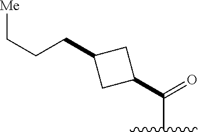 |
| 11 | 700 uM | 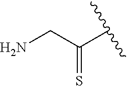 [SMILES: *C(=S)CN] | 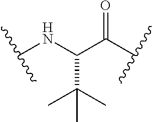 [SMILES: *N[C@H](C(*)=O)C(C)(C)C] | 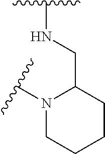 [SMILES: *NCC1CCCCN1*] | 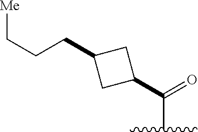 |
| 12 | 1000 uM | 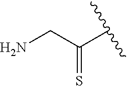 [SMILES: *C(=S)CN] | 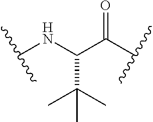 [SMILES: *N[C@H](C(*)=O)C(C)(C)C] | 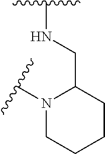 [SMILES: *NCC1CCCCN1*] | 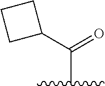 [SMILES: *C(=O)C1CCC1] |
| 13 | 300 uM | 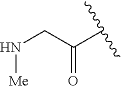 [SMILES: *C(=O)CNC] | 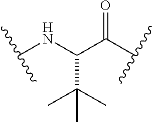 [SMILES: *N[C@H](C(*)=O)C(C)(C)C] | 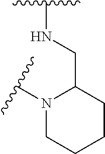 [SMILES: *NCC1CCCCN1*] | 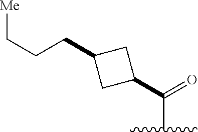 |
| 14 | 250 uM | 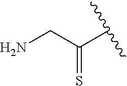 [SMILES: *C(=S)CN] | 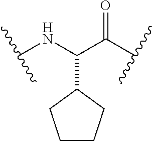 [SMILES: *N[C@H](C(*)=O)C1CCCC1] | 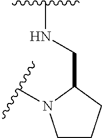 | 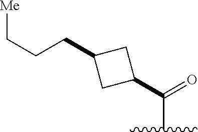 |
| 15 | >3000 nM | 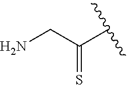 [SMILES: *C(=S)CN] | 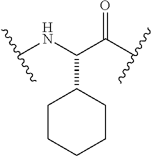 [SMILES: *N[C@H](C(*)=O)C1CCCCC1] | 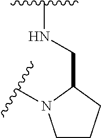 | 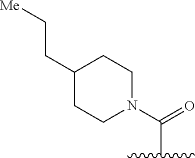 [SMILES: *C(=O)N1CCC(CCC)CC1] |
| 16 | 200 nM | 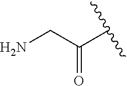 [SMILES: *C(=O)CN] | 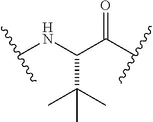 [SMILES: *N[C@H](C(*)=O)C(C)(C)C] | 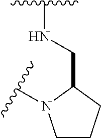 | 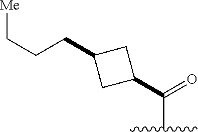 |
| 17 | 100 uM | 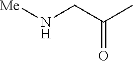 | 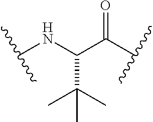 [SMILES: *N[C@H](C(*)=O)C(C)(C)C] | 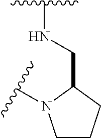 | 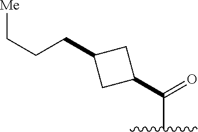 |
| 18 | 20 uM | 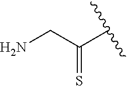 [SMILES: *C(=S)CN] | 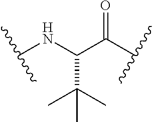 [SMILES: *N[C@H](C(*)=O)C(C)(C)C] | 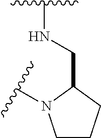 | 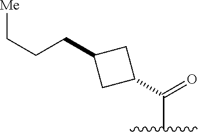 |
| 19 | 2000 nM | 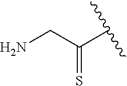 [SMILES: *C(=S)CN] | 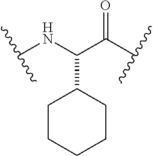 [SMILES: *N[C@H](C(*)=O)C1CCCCC1] | 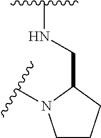 | 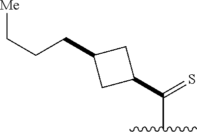 |
| 20 | 650 nM | 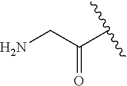 [SMILES: *C(=O)CN] | 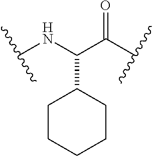 [SMILES: *N[C@H](C(*)=O)C1CCCCC1] | 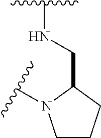 | 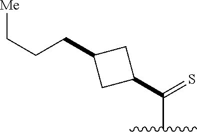 |
| 21 | >3000 nM | H | 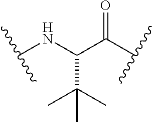 [SMILES: *N[C@H](C(*)=O)C(C)(C)C] | 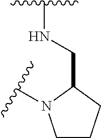 | 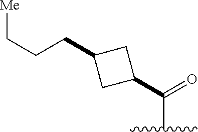 |
| 22 | 30 nM | 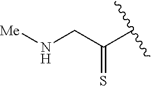 [SMILES: *C(=S)CNC] | 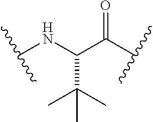 [SMILES: *N[C@H](C(*)=O)C(C)(C)C] | 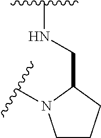 | 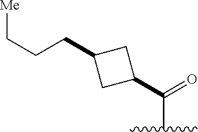 |
| 23 | >3000 nM | 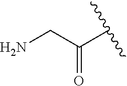 [SMILES: *C(=O)CN] | 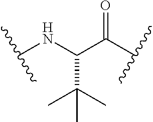 [SMILES: *N[C@H](C(*)=O)C(C)(C)C] | 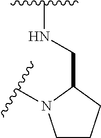 | 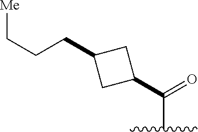 |
| 24 | 2000 nM | 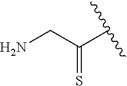 [SMILES: *C(=S)CN] | 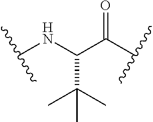 [SMILES: *N[C@H](C(*)=O)C(C)(C)C] | 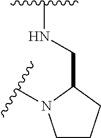 | 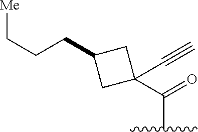 |
| 25 | 35 nM | 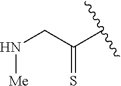 [SMILES: *C(=S)CNC] | 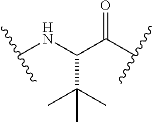 [SMILES: *N[C@H](C(*)=O)C(C)(C)C] | 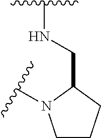 | 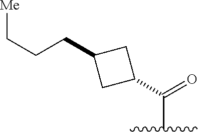 |
| 26 | 25 nM | 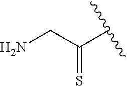 [SMILES: *C(=S)CN] | 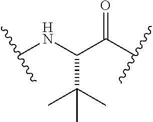 [SMILES: *N[C@H](C(*)=O)C(C)(C)C] | 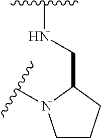 | 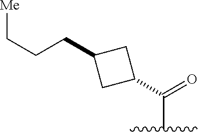 |
| 27 | >300 nM | H | 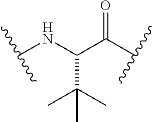 [SMILES: *N[C@H](C(*)=O)C(C)(C)C] | 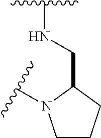 | 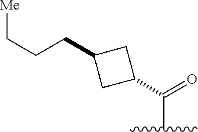 |
| 28 | 70 nM | 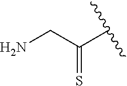 [SMILES: *C(=S)CN] | 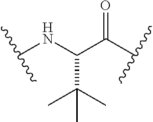 [SMILES: *N[C@H](C(*)=O)C(C)(C)C] | 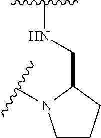 | 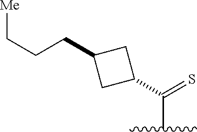 |

A further specific embodiment provides a compound of Formula (IIa2) as

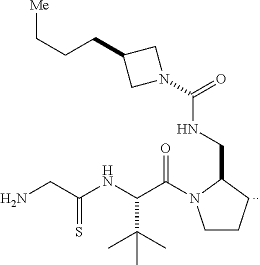

Of the compounds of Formula (II), as set forth above, another embodiment provides a compound wherein:

m is 1;

R¹ is

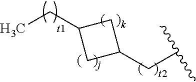

k and j are independently 1, 2 or 3;

t1 and t2 are independently any integer between 0 to 8; and

R⁶ and R⁴ are linked together to form alkylene, alkenylene or alkynylene.

A further specific embodiment provides the following compound:

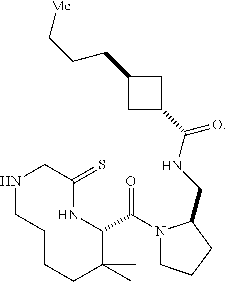

Of the compounds of Formula (II), as set forth above, another embodiment provides a compound wherein:

m is 1;

R¹ is

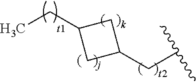

k and j are independently 1, 2 or 3;

t1 and t2 are independently any integer between 0 to 8; and

two adjacent R²s together with the atoms to which they are attached form a 5-, 6- or 7-member heterocyclic or carbocyclic ring.

A further specific embodiment provides the following compound:

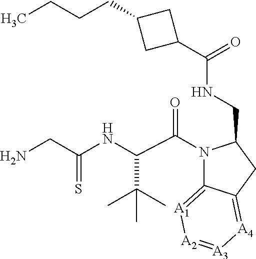

wherein:

A1, A2, A3 and A4 is independently —CH—; —N— or nothing.

Of the compounds of Formula (II), as set forth above, another embodiment provides a compound wherein:

m is 1;

R¹ is

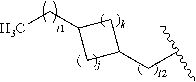

k and j are independently 1, 2 or 3;

t1 and t2 are independently any integer between 0 to 8; and

R⁴ and R² together with the atoms to which they are attached form an N-heterocyclic ring.

A further specific embodiment provides the following compound:

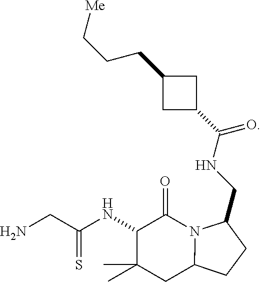

Of the compounds of Formula (II), as set forth above, another embodiment provides a compound wherein:

m is 1;

R¹ is

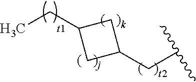

k and j are independently 1, 2 or 3;

t1 and t2 are independently any integer between 0 to 8; and

R⁶ and R¹ are linked together to form alkylene, alkenylene or alkynylene.

A further specific embodiment provides the following compound:

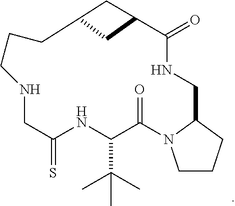 [SMILES: CC(C)(C)[C@@H]1NC(=S)CNCCC[C@H]2C[C@@H](C2)C(=O)NC[C@H]2CCCN2C1=O]

Of the compounds of Formula (II), as set forth above, another embodiment provides a compound of Formula (IIb):

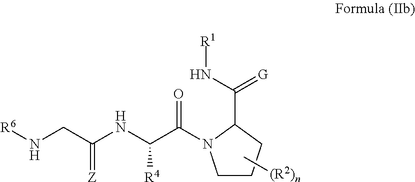

wherein:

n is 0, 1, 2;

G is O or S;

Z is O, S or NH;

R¹ is alkyl or heterocyclyl;

each R² is independently alkyl, aryl, halo, hydroxyl or alkoxy; or

two adjacent R²s together with the atoms to which they are attached form a 5-, 6- or 7-member heterocyclic or carbocyclic ring;

R⁴ is alkyl or cycloalkyl;

R⁶ is hydrogen or alkyl.

Of the compounds of Formula (IIb), as set forth above, another embodiment provides a compound wherein:

R¹ is

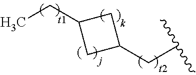

k and j are independently 1, 2 or 3; and

t1 and t2 are independently any integer between 0 to 8.

Of the compounds of Formula (IIb), as set forth above, another embodiment provides a compound represented by Formula (IIb1):

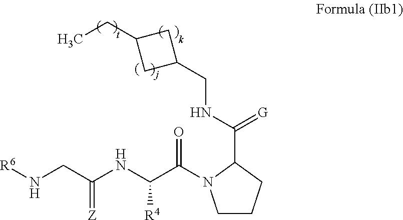

wherein:

k and j are independently 1, 2 or 3;

t is any integer between 0 to 8;

G is O or S;

Z is O, S or NH;

R⁴ is alkyl or cycloalkyl; and

R⁶ is hydrogen or alkyl.

Of the compounds of Formula (IIb), as set forth above, another embodiment provides a compound represented by Formula (IIb2):

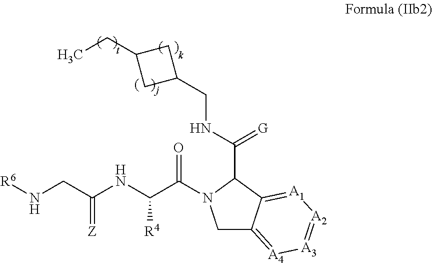

wherein:

k and j are independently 1, 2 or 3;

t is any integer between 0 to 8;

G is O or S;

Z is O, S or NH;

R⁴ is alkyl or cycloalkyl;

R⁶ is hydrogen or alkyl; and

A1, A2, A3 and A4 is independently —CH—; —N— or nothing.

Of the compounds of any one of Formulae (IIb1) or (IIb2), as set forth above, another embodiment provides a compound wherein R⁴ is t-butyl or cyclohexyl.

Of the compounds of any one of Formulae (IIb1) or (IIb2), as set forth above, another embodiment provides a compound wherein Z is S.

Of the compounds of any one of Formulae (IIb1) or (IIb2), as set forth above, another embodiment provides a compound wherein G is O.

A further specific embodiment of Formula (IIb1) provides the following compound:

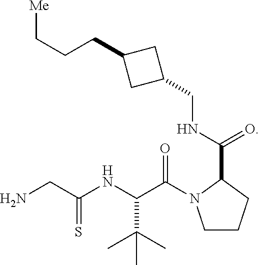

Of the compounds of Formula (II), as set forth above, another embodiment provides a compound represented by Formula (IIc):

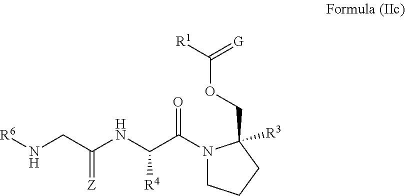

wherein:

G is O or S;

Z is O, S or NH;

R¹ is alkyl or heterocyclyl;

R³ is hydrogen, alkyl, alkenyl or alkynyl;

R⁴ is alkyl or cycloalkyl; and

R⁶ is hydrogen or alkyl.

Of the compounds of Formula (IIc), as set forth above, another embodiment provides a compound wherein R¹ is heterocyclyl.

A further specific embodiment of Formula (IIc) provides the following compound:

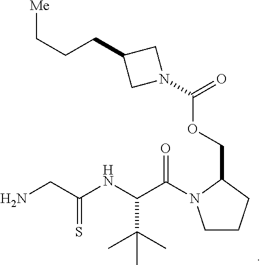

Of the compounds of Formula (I), as set forth above, another embodiment provides a compound wherein:

W is —NR⁹—, —O—, —CH═CH— or —C≡C—;

G is O or S; and

m is 1.

A further embodiment provides a compound wherein R¹ is alkyl.

A further specific embodiment provides a compound wherein R¹ is:

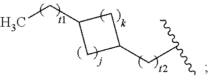

k and j are independently 1, 2 or 3; and

t1 and t2 is independently any integer between 0 to 8.

Further specific embodiments provide the following compounds:

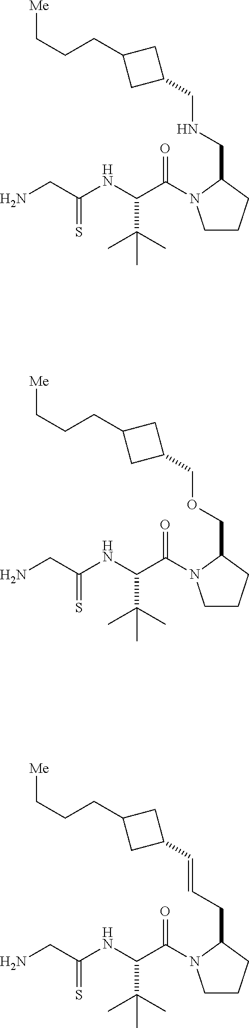

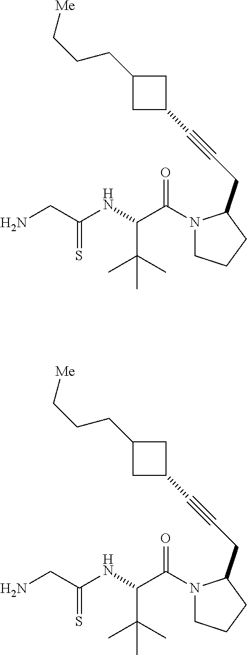

Of the compounds of Formula (I), as set forth above, another embodiment provides a compound wherein:

W is —C(O)—; and

m is 0, 1 or 2.

Of the compounds of the above embodiment, another embodiment provides a compound represented by Formula (III):

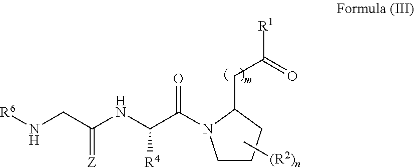

wherein:

n is 0, 1 or 2;

m is 0, 1 or 2;

Z is O, S or NH;

R¹ is alkyl or heterocyclyl;

each R² is independently alkyl, halo, hydroxyl or alkoxy; or

two adjacent R²s together with the atoms to which they are attached form a 5-, 6- or 7-member heterocyclic or carbocyclic ring;

R⁴ is alkyl or cycloalkyl; and

R⁶ is hydrogen or alkyl.

Of the compounds of Formula (III), as set forth above, further specific embodiments provide the following compounds:

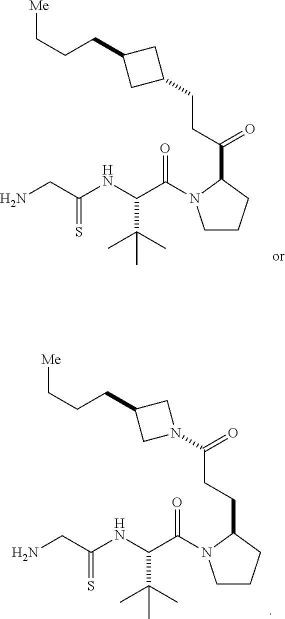

In preferred embodiments, GOAT inhibitors according the present disclosure is:

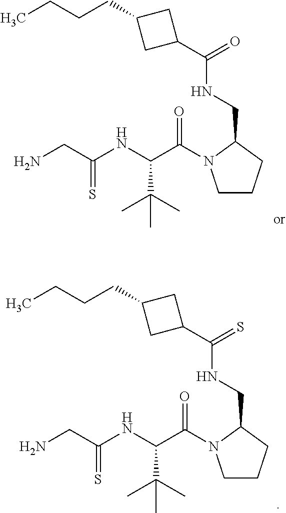

The compounds described herein may generally be used as the free base. Alternatively, the compounds may be used in the form of acid addition salts. Acid addition salts of the free base amino compounds may be prepared according to methods well known in the art, and may be formed from organic and inorganic acids. Suitable organic acids include (but are not limited to) maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include (but are not limited to) hydrochloric, hydrobromic, sulfuric, phosphoric, and nitric acids. Thus, the term “pharmaceutically acceptable salt” of compounds of Formula (I) and substructures thereof, i.e., Formulae (II), (IIa), (IIa1), (IIa2), (IIb), (IIb1), (IIb2), (IIc) and (III), as well as any and all substructures and specific compounds described herein is intended to encompass any and all pharmaceutically suitable salt forms.

In a preferred embodiment, the compound of any one of Formulae (I), (II), (IIa), (IIa1), (IIa2), (IIb), (IIb1), (IIb2), (IIc) and (III) is in the form of a hemitartrate salt.

Furthermore, some of the crystalline forms of any compound described herein, including the salt form, may exist as polymorphs. In addition, some of the compounds may form solvates with water or other organic solvents. Often crystallizations produce a solvate of the disclosed compounds. As used herein, the term “solvate” refers to an aggregate that comprises one or more molecules of any of the disclosed compounds with one or more molecules of solvent. The solvent may be water, in which case the solvate may be a hydrate. Alternatively, the solvent may be an organic solvent. Thus, the presently disclosed compounds may exist as a hydrate, including a monohydrate, dihydrate, hemihydrate, sesquihydrate, trihydrate, tetrahydrate and the like, as well as the corresponding solvated forms.

With regard to stereoisomers, the compounds of any one of Formulae (I), (II), (IIa), (IIa1), (IIa2), (IIb), (IIb1), (IIb2), (IIc) and (III), as well as any substructure herein or specific compounds, may have or have one or more chiral (or asymmetric) centers, and may thus give rise to enantiomers, diastereomers, and other stereoisomeric forms that may be defined, in terms of absolute stereochemistry, as (R)- or (S)-. When the compounds described herein contain olefinic double bonds or other centers of geometric asymmetry, and unless specified otherwise, it is intended that the compounds include both E and Z geometric isomers (e.g., cis or trans). Likewise, unless otherwise specified, all possible isomers, as well as their racemic and optically pure forms, and all tautomeric forms are also intended to be included. It is therefore contemplated that various stereoisomers and mixtures thereof and includes “enantiomers,” which refers to two stereoisomers whose molecules are nonsuperimposable mirror images of one another. Thus, the compounds may occur in any isomeric form, including racemates, racemic mixtures, and as individual enantiomers or diastereomers.

In other embodiments, provided herein are pharmaceutical compositions comprising at least one of the compounds of any one of Formulae (I), (II), (IIa), (IIa1), (IIa2), (IIb), (IIb1), (IIb2), (IIc) and (III) described above and herein and a pharmaceutically acceptable (i.e., suitable) excipient.

### Definitions

“Alkyl” refers to a straight or branched hydrocarbon chain radical or cyclic hydrocarbon radical, when unsubstituted, consisting solely of carbon and hydrogen atoms, containing no unsaturation, and which is attached to the remainder of the molecule by a single bond. The carbon attaching to the remainder of the molecule may be a chain carbon (e.g., methyl) or a ring carbon (e.g., cyclobutyl). Examples of chain radicals include, without limitation, methyl, ethyl, n-propyl, 1-methylethyl (isopropyl), n-butyl, n-pentyl, 1,1-dimethylethyl (t-butyl), and the like. Examples of cyclic radicals include, without limitation, cyclopropyl, cyclobutyl, cyclopentyl, or cyclohexyl, and the like. In certain embodiments, the alkyl may have from one to twenty carbon atoms (C₁₋₂₀), one to twelve carbon atoms (C₁₋₁₂), or preferably one to eight carbon atoms (C₁₋₈) or one to six carbon atoms (C₁₋₆). In other embodiments, the alkyl may have from three to twelve carbon atoms (C₃₋₁₂), or preferably three to eight carbon atoms (C₃₋₈). In certain embodiments, the alkyl radical may contain a mixture of chain hydrocarbon and cyclic hydrocarbon. The carbon attaching to the remainder of the molecule may be the chain carbon or the ring carbon. For example, an alkyl may be cyclopropylmethyl, which is attached to the remainder of the molecule by the methyl, which is further substituted with a cyclic alkyl (i.e., cyclopropyl). Another example of an alkyl may be a butylcyclobutyl, which is attached to the remainder of the molecule by a carbon on the cyclobutyl ring, which is further substituted by a butyl. In these embodiments, the number of carbons in an alkyl counts both the chain and cyclic carbons, and may have up to 20 carbons, or may have four to twelve carbons (C₄₋₁₂), or may have four to eight carbons (C₄-8), or 4, 5, 6, 7, 8, 9, 10, 11 or 12 carbons. Unless stated otherwise specifically in the specification, an alkyl group may be unsubstituted or substituted by one or more substituents, as defined herein.

“Alkylene” refers to a straight or branched hydrocarbon divalent radical linking two portions of a compound according to the present disclosure, consisting solely of carbon and hydrogen, containing no unsaturation and having from one to twenty carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. An alkylene chain may also include, within the chain, a cycloalkyl ring.

“Alkenyl” refers to an alkyl radical with at least one unsaturation, i.e., at least one C=C either in the chain hydrocarbon radical or the ring hydrocarbon radical.

“Alkenylene” refers to a straight or branched hydrocarbon divalent radical linking two portions of a compound according to the present disclosure, consisting solely of carbon and hydrogen and at least one C═C bond, and having from one to twenty carbon atoms. The alkenylene chain is attached to the rest of the molecule through a single bond and to the radical group through a single bond. The points of attachment of the alkenylene chain to the rest of the molecule and to the radical group can be through one carbon or any two carbons within the chain. An alkenylene chain may also include, within the chain, a cycloalkyl ring.

“Alkynyl” refers to an alkyl radical with least one C≡C in the chain hydrocarbon radical.

“Alkynylene” refers to a straight or branched hydrocarbon divalent radical linking two portions of a compound according to the present disclosure, consisting solely of carbon and hydrogen and at least one C≡C bond, and having from one to twenty carbon atoms. The alkynylene chain is attached to the rest of the molecule through a single bond and to the radical group through a single bond. The points of attachment of the alkynylene chain to the rest of the molecule and to the radical group can be through one carbon or any two carbons within the chain. An alkynylene chain may also include, within the chain, a cycloalkyl ring.

“Alkoxy” refers to the radical —O-alkyl, wherein alkyl is as defined herein. C₁₋₃alkoxy refers to alkoxy having 1-3 carbon chain atoms, e.g., methoxy, ethoxy, and the like.

“Alkyloxycarbonyl” refers to an alkyl-O—C(O)— radical.

“Alkenyloxycarbonyl” refers to an alkenyl-O—C(O)— radical.

“Halo” refers to bromo, chloro, fluoro or iodo.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to, phenyl and naphthyl. Unless stated otherwise specifically in the specification, an alkyl group may be unsubstituted or substituted by one or more substituents, as defined herein.

“Aralkyl” refers to an alkyl radical, as defined herein, which is further substituted with an aryl, as defined herein.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, and the like. Unless otherwise stated specifically in the specification, the term “cycloalkyl” is meant to include cycloalkyl radicals which are optionally substituted by one or more substituents, as defined herein.

“Carbocyclic ring” refers to a ring structure consisting solely of carbon and hydrogen atoms, which may include fused or bridged ring systems, having from three to fifteen carbon atoms, preferably having from three to ten carbon atoms, and which is saturated or unsaturated. Saturated monocyclic ring include, for example, cyclopropyl, cyclobutyl, cyclopentyl, cyclohexyl, cycloheptyl, and cyclooctyl. Unsaturated monocyclic ring includes a phenyl ring. A carbocyclic ring is typically fused to the rest of the molecule by two points of attachments. Unless otherwise stated specifically in the specification, the term “carbocyclic ring” is meant to include carbocyclic rings which are optionally substituted by one or more substituents, as defined herein.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. A heteroaryl may be attached to the remainder of the molecule by a carbon or a heteroatom (e.g., nitrogen). An example of heteroaryl is indolyl, indazolyl, isoindolyl, indolinyl, isoindolinyl, isoquinolyl, indolizinyl, isoxazolyl and the like. Unless stated otherwise specifically in the specification, an alkyl group may be unsubstituted or substituted by one or more substituents, as defined herein.

“Heteroarylalkyl” refers to an alkyl radical, as defined herein, which is further substituted with a heteroaryl, as defined herein.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. A heterocyclyl may be attached to the remainder of the molecule by a carbon or a heteroatom (e.g., nitrogen). Examples of such heterocyclyl radicals include, but are not limited to, dioxolanyl, thienyl[1,3]dithianyl, decahydroisoquinolyl, imidazolinyl, and the like. Unless stated otherwise specifically in the specification, an alkyl group may be unsubstituted or substituted by one or more substituents, as defined herein.

“Heterocyclylalkyl” refers to an alkyl radical, as defined herein, which is further substituted with a heterocyclyl, as defined herein.

“Heterocyclic ring” refers to a ring structure comprising carbon and hydrogen atoms, and at least one heteroatom, which may include fused or bridged ring systems, having from three to fifteen carbon atoms, preferably having from one to seven carbon atoms, and which is saturated or unsaturated. A heterocyclic ring is typically fused to the rest of the molecule by two points of attachments. Unless otherwise stated specifically in the specification, the term “heterocyclic ring” is meant to include carbocyclic rings which are optionally substituted by one or more substituents, as defined herein.

“Substituent” refers to alkyl, alkoxy, halo, haloalkyl (alkyl substituted with one or more halo), cyano (—CN), oxo (═O), nitro (—NO₂), aryl, cycloalkyl, heterocyclyl, heterocyclylalkyl, heteroaryl, —R¹⁵—OR¹⁴, —R⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, aryl, heterocyclyl, heteroaryl; each R¹⁵ is independently a direct bond or a straight or branched alkylene or alkenylene chain; and each R¹⁶ is hydrogen, alkyl, haloalkyl, cycloalkyl, aryl, heterocyclyl, heteroaryl.

“Prodrug” is meant to indicate a compound that may be converted under physiological conditions or by solvolysis to a biologically active compound described herein. Thus, the term “prodrug” refers to a metabolic precursor of a compound described herein that is pharmaceutically acceptable. A prodrug may be inactive when administered to a subject in need thereof, but is converted in vivo to an active compound as described herein. Prodrugs are typically rapidly transformed in vivo to yield the parent compound described herein, for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers which release the active compound as described herein in vivo when such prodrug is administered to a mammalian subject. Prodrugs of a compound described herein may be prepared by modifying functional groups present in the compound described herein in such a way that the modifications are cleaved, either in routine manipulation or in vivo, to the parent compound described herein. Prodrugs include compounds described herein wherein a hydroxy, amino or mercapto group is bonded to any group that, when the prodrug of the compound is administered to a mammalian subject, cleaves to form a free hydroxy, free amino or free mercapto group, respectively. Examples of prodrugs include, but are not limited to, ester and amide derivatives of hydroxy, carboxy, mercapto or amino functional groups in the compounds described herein and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substituents on the functional group are also “optionally substituted” and so on, for the purposes of this invention, such iterations are limited to five.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“Pharmaceutically acceptable excipient, carrier, or diluent” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Stable compound” and “stable structure” are meant to indicate a compound that is sufficiently robust to survive isolation to a useful degree of purity from a reaction mixture, and formulation into an efficacious therapeutic agent.

“Mammal” includes humans, and also includes domesticated animals such as laboratory animals, livestock and household pets (e.g., cats, dogs, swine, cattle, sheep, goats, horses, rabbits), and also includes non-domesticated animals such as wildlife and the like.

“Therapeutically effective amount” refers to that amount of a compound of the disclosure which, when administered to a mammal, preferably a human, is sufficient to effect treatment, as defined below, of a disease or condition in the mammal, preferably a human. The amount of a compound of the disclosure which constitutes a “therapeutically effective amount” will vary depending on the compound, the condition and its severity, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or disorder of interest, and includes:

(i) preventing from occurring, in a mammal, or reducing in a statistically significant manner (e.g., relative to appropriate controls) the likelihood of occurrence or severity of the disease or condition in a mammal, in particular, when such mammal is predisposed to the condition but has not yet been diagnosed as having it;

(ii) inhibiting the disease or condition, i.e., arresting its development, for instance, preventing progression of a pre-diabetic condition from an early defined stage such as impaired glucose tolerance to a more advanced defined stage such as insulin resistance, or attenuating progression (e.g., decreasing the frequency of such progression events in a statistically significant manner, or increasing the timeframe in which such progression occurs in a statistically significant manner), or interfering with disease progression to completely or partially block or attenuate diabetes, for instance, to substantially impair disease progression, which may refer to substantial and statistically significant, but not necessarily complete, inhibition of progression, e.g., at least 50%, 60%, 70%, 80%, 85%, 90%, 95% or greater inhibition relative to appropriate untreated controls;

(iii) relieving the disease or condition, i.e., causing regression of the disease or condition; or

(iv) relieving the symptoms resulting from the disease or condition, e.g., halting or reversing weight gain without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. In preferred embodiments the present compositions and methods will find uses in the treatment of a condition that would benefit from a decreased (e.g., reduced in a statistically significant manner relative to an untreated control) level of acylated ghrelin peptide. Accordingly and in certain preferred embodiments, the present disclosure contemplates methods of treating type 2 diabetes, obesity, or a related disease, disorder, metabolic dysregulation or other condition.

### Methods of Use and Pharmaceutical Compositions

Provided herein are methods of treatment using the herein disclosed compounds that dramatically lower the availability of active ghrelin, by inhibiting the GOAT-mediated acylation of ghrelin by which the predominantly active form of ghrelin (i.e., octanoylated ghrelin) is generated. In one embodiment, a compound of the present invention is administered to a patient having a disease, disorder or condition involving inappropriate, excessive or otherwise deleterious ghrelin activity mediated by active (i.e., octanoylated) ghrelin, the activity level of which may be altered (e.g., decreased in a statistically significant manner) by blocking GOAT-catalyzed enzymatic ghrelin octanoylation. In the context of the present disclosure such a disease, disorder or condition includes diseases and disorders characterized by aberrant ghrelin activity, due for example to alterations (e.g., statistically significant increases or decreases) in the amount or activity of active ghrelin or of a ghrelin-interacting molecule (e.g., a cellular receptor to which active ghrelin specifically binds to mediate a biological effect such as signal transduction) that is present, or the presence of a mutant ghrelin-interacting molecule, or both. Certain presently contemplated embodiments therefore relate to a method for treating a subject known to have, or suspected of being at risk for having, a condition that would benefit from a decreased level of acylated (e.g., octanoylated) ghrelin peptide, which method comprises administering to the subject a therapeutically effective amount of at least one of the herein disclosed GOAT-inhibiting compounds.

An overabundance of active ghrelin may be due to any cause, including but not limited to overexpression at the molecular level, prolonged or accumulated appearance at the site of action, or increased (e.g., in a statistically significant manner) activity of ghrelin relative to that which is normally detectable. Such an overabundance of ghrelin activity can be measured relative to normal expression, appearance, or activity of ghrelin and said measurement may play an important role in the development and/or clinical testing of the compounds described herein.

In particular, the presently disclosed compounds are useful for the treatment of diabetes and specifically certain complications of diabetes, by inhibiting GOAT-mediated ghrelin octanoylation and thus impeding subsequent events that depend on active (octanoylated) ghrelin. Thus, in certain embodiments, the compounds described herein are useful for the treatment of diseases associated with diabetes including type 2 diabetes, such as, impaired glucose tolerance, insulin resistance, or other related disorders or conditions, including associated symptoms, hypercholesterolemia, hypertriglyceridemia, cardiovascular disease, hypertension, nephropathy, retinopathy and neuropathy.

In type II diabetes, resistance to insulin results in the lack of glucose uptake by tissues such as skeletal muscles. The insulin-resistance results in higher blood glucose levels, and the pancreas produces more insulin to compensate for the higher blood glucose levels. Insulin resistance may be diagnosed via a hyperinsulinemic-euglycemic clamp. The GOAT-inhibitor compounds of certain of the instant invention embodiments may be administered to diabetic patients exhibiting insulin resistance.

In certain embodiments where it is desirable to determine whether or not a subject (e.g., a patient) presents within clinical parameters indicative of type 2 diabetes mellitus, signs and symptoms of type 2 diabetes that are accepted by those skilled in the art may be used to so designate the subject, for example, the clinical signs referred to in Gavin et al. (Diabetes Care 22(suppl. 1):S5-S19, 1999, American Diabetes Association Expert Committee on the Diagnosis and Classification of Diabetes Mellitus) and references cited therein, or other means known in the art for diagnosing type 2 diabetes.

In diabetes and certain other metabolic diseases or disorders, one or more biochemical processes, which may be either anabolic or catabolic (e.g., build-up or breakdown of substances, respectively), are altered (e.g., increased or decreased in a statistically significant manner) or modulated (e.g., up- or down-regulated to a statistically significant degree) relative to the levels at which they occur in a disease-free or normal subject such as an appropriate control individual. The alteration may result from an increase or decrease in a substrate, enzyme, cofactor, or any other component in any biochemical reaction involved in a particular process. An extensive set of altered indicators of mitochondrial function, for example, has been described for use in determining the presence of, and characterizing, diabetes (see, e.g., U.S. Pat. No. 6,140,067).

Accordingly in these and related embodiments there is provided a method of inhibiting GOAT enzyme catalytic activity (e.g., acylation such as octanoylation of a ghrelin polypeptide such as des-acyl ghrelin, for instance, the murine ghrelin peptide GSSFLSPEHQKAQQRKESKKPPAKLQPR [SEQ ID NO:1] or the human ghrelin peptide GSSFLSPEHQRVQQRKESKKPPAKLQPR [SEQ ID NO:2] or any other vertebrate ghrelin peptide such as one or more of the peptides having the amino acid sequences set forth in SEQ ID NOS:1-9 or disclosed in Kojima et al., 2005 Physiol. Rev. 85:495-522), comprising contacting a composition which comprises a catalytically active GOAT enzyme and a herein-described GOAT inhibitor (e.g., a compound having a structure that is within one of the structures of Formula (I) and substructures thereof, i.e., Formulae (II), (IIa), (IIa1), (IIa2), (IIb), (IIb1), (IIb2), (IIc) and (III), as well as any and all substructures and specific compounds described herein as provided herein) under conditions and for a time sufficient for the GOAT inhibitor to interact specifically with the GOAT enzyme, and to inhibit GOAT-mediated acylation of ghrelin. As described herein in the illustrative examples, such contacting may typically involve a method whereby the GOAT polypeptide and the GOAT inhibitor are afforded an opportunity physically to contact one another (e.g., by exposing, introducing, admixing, incubating or otherwise bringing into close and unhindered proximity), and these and related embodiments further contemplate determining inhibition of the acylation activity of the GOAT enzyme, for example, by detecting a level of enzymatic acylation by GOAT of a detectable substrate (e.g., incorporation of radiolabeled octanoylate into a ghrelin peptide) in the absence of the GOAT inhibitor that differs (with statistical significance) from the level of enzymatic incorporation of radiolabeled octanoylate by GOAT into ghrelin in the presence of the GOAT inhibitor.

Operable conditions, including solution conditions, temperature, and incubation times, for determining GOAT activity such as the ability of a GOAT enzyme to catalyze octanoylation of a des-acyl ghrelin peptide substrate as provided herein (e.g., SEQ ID NOS:1-9 or any other vertebrate ghrelin peptide such as one or more of the peptides having the amino acid sequences set forth in SEQ ID NOS:1-9 or disclosed in Kojima et al., 2005 Physiol. Rev. 85:495-522) are known to persons familiar with the art (e.g., Yang et al., 2008 Proc. Nat. Acad. Sci. USA 105:10750) and/or can be readily identified using only routine experimentation, based on existing knowledge in enzymology generally, and specifically with regard to peptide O-acyl transferases. These and related embodiments may afford identification from amongst the presently disclosed GOAT inhibitors of those having particularly desirable properties, depending on intended uses such as, e.g., formulations for particular routes of administration or having one or more GOAT inhibitors of particular efficacies, potencies and/or physicochemical or pharmacokinetic properties.

As also described herein, the compounds identified herein as GOAT inhibitors exhibit significant selectivity toward GOAT as contrasted with inhibitory activity against other acyl transferases, such that any specific one of the present GOAT inhibitors should have an IC₅₀ value when tested against GOAT that is lower (i.e., in a statistically significant manner) than the IC₅₀ value when tested against an unrelated acyl transferase of irrelevant substrate specificity. Preferred are GOAT inhibitors that exhibit at least 10-fold, 20-fold, 30-fold, 40-fold, 50-fold, 60-fold, 70-fold, 75-fold, 80-fold, 100-fold, 500-fold, 1000-fold or greater selectivity for GOAT relative to an unrelated acyl transferase. A GOAT inhibitor as described herein preferably substantially impairs GOAT-mediated octanoylation of des-acyl ghrelin (e.g., a peptide having the sequence set forth in one of SEQ ID NOS:1-9 or any other vertebrate ghrelin peptide such as those disclosed in Kojima et al., 2005 Physiol. Rev. 85:495-522),), which may refer to substantial and statistically significant, but not necessarily complete, inhibition of O-octanoylation of ghrelin at the serine residue in position number three of SEQ ID NO:1, e.g., at least 50%, 60%, 70%, 80%, 85%, 90%, 95%, 96%, 97%, 98%, 99% or greater inhibition of ghrelin acylation relative to the amount of ghrelin acylation that occurs in an appropriate control reaction (e.g., with no inhibitor present or with a negative control inhibitor such as a compound known to be incapable of inhibiting GOAT).

Mammalian GOAT is a well known polypeptide for which catalytic activity has been characterized using cell membrane preparations prepared from GOAT-expressing cells (e.g., U.S. application Ser. No. 12/167,917; Yang et al. 2008 Cell 132:387; Gutierrez et al., 2008 Proc. Nat. Acad. Sci. USA 105:6320; Yang et al. 2008 Proc. Nat. Acad. Sci. USA 105:10750; Taylor et al., 2012 Meth. Enzymol. 514:205; see also U.S. Pat. No. 7,544,466.), including cells that have been recombinantly engineered to express GOAT.

A GOAT polypeptide for use in certain embodiments contemplated herein may therefore comprise the amino acid sequence set forth in any one of Genbank Accession (gene ID) Nos. 234155 (murine GOAT), 619373 (human GOAT), 100529112 (canine GOAT), or 306515 (rat GOAT) and, and may in certain other embodiments comprise a GOAT polypeptide variant comprising a polypeptide that is at least 85%, 90%, 95%, 96%, 97%, 98% or 99% identical to such polypeptides and that is capable of specific enzymatic octanoylation of a des-acyl ghrelin peptide such as the peptide having the amino acid sequence set forth in any one or more of SEQ ID NOS:1-9 or any other vertebrate ghrelin peptide such as those disclosed in Kojima et al., 2005 Physiol. Rev. 85:495-522. In certain other embodiments a GOAT polypeptide may comprise a polypeptide that comprises a GOAT catalytic domain or a functional fragment thereof or variant thereof, the GOAT catalytic domain or functional fragment thereof or variant thereof comprising an amino acid sequence that is at least 80%, 85%, 90% or 95% identical to the amino acid sequence set forth in any one of Genbank Accession (gene ID) Nos. 234155 (murine GOAT), 619373 (human GOAT), 100529112 (canine GOAT), or 306515 (rat GOAT) and that is capable of specific enzymatic octanoylation of a des-acyl ghrelin peptide such as the peptide having the amino acid sequence set forth in any one or more of SEQ ID NOS:1-9 or any other vertebrate ghrelin peptide such as those disclosed in Kojima et al., 2005 Physiol. Rev. 85:495-522.

Polypeptide variants of a GOAT polypeptide or of a GOAT catalytic domain or a functional fragment thereof may contain one or more amino acid substitutions, additions, deletions, and/or insertions relative to a native GOAT polypeptide sequence such as the amino acid sequence set forth in any one of Genbank Accession (gene ID) Nos. 234155 (murine GOAT), 619373 (human GOAT), 100529112 (canine GOAT), and 306515 (rat GOAT) (e.g. wildtype, or a predominant or naturally occurring allelic form). Variants preferably exhibit at least about 75%, 78%, 80%, 85%, 87%, 88% or 89% identity and more preferably at least about 90%, 92%, 95%, 96%, 97%, 98%, or 99% identity to a portion of a native GOAT polypeptide sequence. The percent identity may be readily determined by comparing sequences of the polypeptide variants with the corresponding portion of a full-length polypeptide.

Some techniques for sequence comparison include using computer algorithms well known to those having ordinary skill in the art, such as Align or the BLAST algorithm (Altschul, J. Mol. Biol. 219:555-565, 1991; Henikoff and Henikoff, PNAS USA 89:10915-10919, 1992), which is available at the NCBI website (see [online] Internet:<URL: http://www/ncbi.nlm.nih.gov/cgi-bin/BLAST). Default parameters may be used.

Furthermore, computer algorithms are available in the art that enable the skilled artisan to predict the three-dimensional structure of a protein or peptide, in order to ascertain functional variants of a particular polypeptide. For instance, variants can be identified wherein all or a portion of the three-dimensional structure is not substantially altered by one or more modification, substitution, addition, deletion and/or insertion. (See, for example, Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007)). In this way, one of skill in the art can readily determine whether a particular GOAT polypeptide variant or a GOAT domain or a functional fragment thereof is capable of specific enzymatic octanoylation of a des-acyl ghrelin peptide such as the peptide having the amino acid sequence set forth in any one or more of SEQ ID NOS:1-9 or any other vertebrate ghrelin peptide such as those disclosed in Kojima et al., 2005 Physiol. Rev. 85:495-522.

Methodologies for the design, production and testing of GOAT polypeptides and polypeptide variants and of GOAT catalytic domains and functional fragments thereof as provided herein are all available by minor modification to existing knowledge in the art, for example, using conventional methods of virology, immunology, microbiology, molecular biology and recombinant DNA techniques, which are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

### Pharmaceutical Compositions and Administration

The present invention also relates in certain embodiments to pharmaceutical compositions containing the compounds of the invention disclosed herein. In one embodiment, the present invention relates to a pharmaceutical composition comprising compounds of the invention in a pharmaceutically acceptable excipient, carrier or diluent and in an amount effective to confer benefit for a condition that would benefit from a decreased level of acylated ghrelin peptide when administered to an animal, preferably a mammal, most preferably a human.

Administration of the compounds disclosed herein, or their pharmaceutically acceptable salts, in pure form or in an appropriate pharmaceutical composition, can be carried out via any of the accepted modes of administration of agents for serving similar utilities. The pharmaceutical compositions can be prepared by combining a herein disclosed compound with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, intranasal, intraperitoneal, intravenous, intraarterial, transdermal, sublingual, subcutaneous, intramuscular, rectal, transbuccal, intranasal, liposomal, via inhalation, intraocular, via local delivery, subcutaneous, intraadiposal, intraarticularly or intrathecally. The term parenteral as used herein includes subcutaneous injections, intravenous, intramuscular, intrasternal injection or infusion techniques. Pharmaceutical compositions are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a herein disclosed compound in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a herein disclosed compound, or a pharmaceutically acceptable salt thereof, for treatment of a disease or condition of interest in accordance with the teachings of this disclosure.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition according to certain embodiments of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel™, corn starch and the like; lubricants such as magnesium stearate or Sterotex™; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should according to certain embodiments contain an amount of a herein disclosed compound such that a suitable dosage will be obtained. Typically, this amount is at least 0.01% of a herein disclosed compound in the composition. When intended for oral administration, this amount may be varied to be between 0.1 and about 70% of the weight of the composition. Preferred oral pharmaceutical compositions contain between about 4% and about 50% of the compound. Preferred pharmaceutical compositions and preparations according to the present invention are prepared so that a parenteral dosage unit contains between 0.01 to 10% by weight of the compound prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of a herein disclosed compound of from about 0.1 to about 10% w/v (weight per unit volume).

The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition in solid or liquid form may include an agent that binds to a herein disclosed compound and thereby assists in the delivery of the compound. Suitable agents that may act in this capacity include a monoclonal or polyclonal antibody, a protein or a liposome.

The pharmaceutical composition may consist of dosage units that can be administered as an aerosol. The term aerosol is used to denote a variety of systems ranging from those of colloidal nature to systems consisting of pressurized packages.

Delivery may be by a liquefied or compressed gas or by a suitable pump system that dispenses the active ingredients. Aerosols of the herein disclosed compounds may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions may be prepared by methodology well known in the pharmaceutical art. For example, a pharmaceutical composition intended to be administered by injection can be prepared by combining a herein disclosed compound with sterile, distilled water so as to form a solution. A surfactant may be added to facilitate the formation of a homogeneous solution or suspension. Surfactants are compounds that non-covalently interact with the compound so as to facilitate dissolution or homogeneous suspension of the compound in the aqueous delivery system.

The compounds according to certain embodiments of the invention disclosed herein, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered in a single dose or by multiple doses over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of the herein disclosed compounds and/or compositions can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition to a target area, e.g., skin surfaces, mucous membranes, and the like. This amount will generally range from about 0.0001 mg to about 1 g of a herein disclosed compound per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the herein disclosed compounds as desired.

The pharmaceutical compositions can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia 22 (6): 543-551 (1997), all of which are incorporated herein by reference.

The compositions can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devices used to apply testosterone as described in PCT Published Patent No. WO 98/50016.

Current methods for ocular delivery include topical administration (eye drops), subconjunctival injections, periocular injections, intravitreal injections, surgical implants and iontophoresis (uses a small electrical current to transport ionized drugs into and through body tissues). Those skilled in the art would combine the best suited excipients with the compound for safe and effective intra-ocular administration.

The most suitable route will depend on the nature and severity of the condition being treated. Those skilled in the art are also familiar with determining administration methods (e.g. oral, intravenous, inhalation, sub-cutaneous, rectal, etc.), dosage forms, suitable pharmaceutical excipients and other matters relevant to the delivery of the compounds to a subject in need thereof.

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

Unless the context requires otherwise, throughout the present specification and claims, the word “comprise” and variations thereof, such as, “comprises” and “comprising” are to be construed in an open, inclusive sense, that is, as “including, but not limited to”. By “consisting of” is meant including, and typically limited to, whatever follows the phrase “consisting of.” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and may or may not be present depending upon whether or not they affect the activity or action of the listed elements.

In this specification and the appended claims, the singular forms “a,” “an” and “the” include plural references unless the content clearly dictates otherwise.

As used herein, in particular embodiments, the terms “about” or “approximately” when preceding a numerical value indicates the value plus or minus a range of 5%, 6%, 7%, 8% or 9%. In other embodiments, the terms “about” or “approximately” when preceding a numerical value indicates the value plus or minus a range of 10%, 11%, 12%, 13% or 14%. In yet other embodiments, the terms “about” or “approximately” when preceding a numerical value indicates the value plus or minus a range of 15%, 16%, 17%, 18%, 19% or 20%.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### EXAMPLES

### I. Compound Preparation

The compounds of any one of Formulae (I), (II), (IIa), (IIa1), (IIa2), (IIb), (IIb1), (IIb2), (IIc) and (III) may be synthesized according to the methods described herein.

Lead GOAT inhibitor 1 is prepared in nine steps beginning with known monoprotected diamine 8 (Jin, Z. et al., Technology Dev Shanghai Co Ltd., Shanghai Inst. Org. Chem, assignees. Preparation method of (R) or (S)-2-aminomethyl tetrahydropyrrole, China patent CN20091055298 20090724, 2011 Feb. 2) and trans-3-butylcyclobutanecarboxylic acid 9. Brannock, K. C. et al., 1964, J. Org. Chem. 29:801-812; Dehmlow, E. V. et al., 1990, Liebigs Annalen der Chemie, 1990:411-414. Closely related and equally potent inhibitor 10 can be prepared in seven steps from the same materials, and negative control 2 is prepared in four steps. The route to 1 is slightly longer due to the need for carbamoyl protection of the primary amine to achieve a selective monothionation reaction. Characterization of the hemitartrate forms of 1, 2, and 10 are shown in Example 1. Full experimental details for the preparation of 10 are provided in Examples 2-3.

### Example 1

### Spectroscopic Characterization of Goat Inhibitors

### Example 1.1

### (1s,3R)-3-BUTYL-N—(((R)-1-((S)-3,3-DIMETHYL-2-(2-(METHYLAMINO)ETHANETHIOAMIDO)BUTANOYL)PYRROLIDIN-2-YL)METHYL)CYCLOBUTANE CARBOXAMIDE HEMITARTRATE (1)

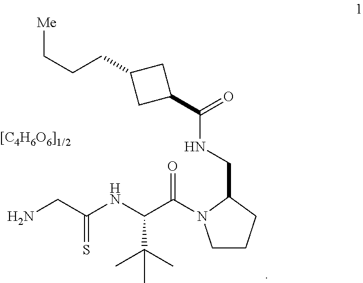

¹H NMR (500 MHz, DMSO) δ: 7.56 (t, J=5.7, 1H), 5.18 (2, 2H), 4.04 (s, 1H), 3.96 (app sextet, J=4.5, 1H), 3.83 (ddd, J=10.2, 8.4, 3.3, 1H), 3.64 (s, 2H), 3.56 (ddd, 9.4, 8.0, 8.0, 1H), 3.19 (ddd, J=12.8, 4.7, 4.7, 1H), 3.03 (ddd, J=13.0, 8.7, 6.9, 1H), 2.91-2.84 (m, 1H), 2.31 (s, 3H), 2.21-2.09 (m, 3H), 1.93-1.79 (m, 2H), 1.75-1.61 (m, 4H), 1.36 (dt, J=8.0, 7.0, 2H), 1.23 (app sextet, J=7.3, 2H), 1.16-1.09, (m, 2H), 0.98 (s, 9H), 0.84 (t, J=7.2, 3H). ¹³C NMR (125 MHz, DMSO) δ: 199.6, 175.3, 174.2, 168.0, 72.1, 62.4, 60.7, 57.4, 47.9, 36.10, 36.01, 35.9, 35.4, 31.8, 30.39, 30.25, 29.4, 27.5, 27.2, 26.9, 25.5, 22.6, 14.5.

### Example 1.2

### (1s,3R)—N—(((R)-1-((S)-2-AMINO-3,3-DIMETHYLBUTANOYL)PYRROLIDIN-2-YL)METHYL)-3-BUTYLCYCLOBUTANECARBOXAMIDE HEMITARTRATE (2)

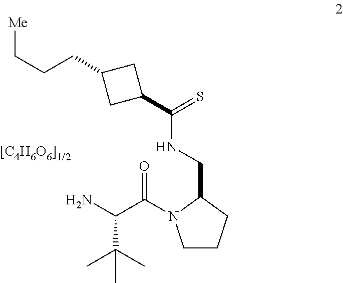

¹H NMR (500 MHz, DMSO) δ: 7.70 (t, J=5.9, 1H), 4.01-3.92 (m, 1H), 3.87 (s, 3H), 3.58-3.50 (m, 2H), 3.45 (dt, J=10.2, 7.6, 1H), 3.29 (dt, J=12.9, 4.7, 1H), 3.23-3.12 (m, 2H), 2.97-2.90 (m, 1H), 2.20-2.12 (m, 3H), 1.90-1.67 (m, 6H), 1.37 (dt, J=8.0, 7.0, 2H), 1.23 (app sextet, 7.2, 2H), 1.17-1.10 (m, 2H), 0.93 (s, 9H), 0.83 (t, J=7.3, 3H). 13C NMR (125 MHz, DMSO) δ: 175.5, 174.8, 170.0, 71.9, 58.9, 57.4, 47.7, 36.0, 35.9, 34.4, 31.8, 30.35, 30.30, 29.4, 27.5, 26.9, 26.6, 23.7, 22.6, 14.5.

### Example 1.3

### (1s,3R)—N—(((R)-1-((S)-2-(2-AMINOETHANETHIOAMIDO)-3,3-DIMETHYLBUTANOYL)PYRROLIDIN-2-YL)METHYL)-3-BUTYLCYCLOBUTANECARBO-THIOAMIDE HEMITARTRATE (10)

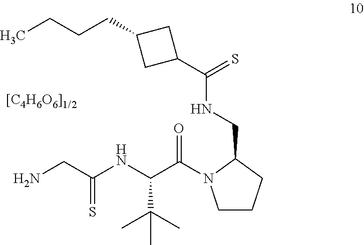

¹H NMR (500 MHz, DMSO) δ: 9.65 (bt, J=5.2, 1H), 7.03 (bs, 3H), 5.20 (s, 2H), 4.22 (ddd, J=14.6, 7.6, 1.34, 1H), 3.94 (s, 1H), 3.89 (ddd, J=10.9, 8.5, 2.4, 1H), 3.68 (d, J=2.2, 2H), 3.65-3.52 (m, 3H), 3.45-3.30 (m, 1H), 2.39-2.30 (m, 2H), 2.2-2.07 (m, 2H), 1.98-1.68 (m, 6H), 1.42 (quartet, J=7.4, 2H), 1.24 (pentet, J=7.2, 2H), 1.19-1.12 (m, 2H), 1.00 (s, 9H), 0.84 (t, J=7.3, 3H), 13C NMR (125 MHz, DMSO) δ: 208.7, 200.7, 174.6, 168.7, 71.8, 62.7, 56.5, 50.7, 48.0, 46.8, 43.7, 36.2, 35.7, 33.13, 33.09, 30.5, 29.6, 21.9, 26.9, 23.7, 22.6, 14.5.

### Example 2

### SYNTHESIS OF CARBOXYLIC ACID 9

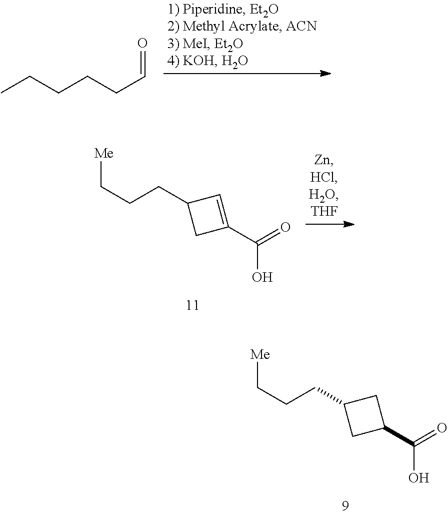

### Example 2.1

### 3-BUTYLCYCLOBUT-1-ENECARBOXYLIC ACID (11)

Piperidine (20 mL, 202.4 mmol) was dissolved in ether (75 mL) containing 4 A molecular sieves (50 g). The solution was cooled in an ice bath, and hexanal (25 mL, 202.4 mmol) was added with stirring over 5 minutes. Stirring was stopped and the solution was allowed to warm to room temperature and maintained at this temperature overnight. The solution was filtered over celite (previously dried in an oven overnight), the filter cake washed with additional ether, and the solvent removed in vacuo. The resulting colorless oil was dissolved in acetonitrile (75 mL) and methyl acrylate (18.22 mL, 202.4 mmol) was added. A reflux condenser was placed on the flask, and the biphasic solution was heated at reflux for three hours. During this time the solution turned orange and became monophasic. The reaction mixture was then cooled and concentrated in vacuo. The resulting thick orange oil was dissolved in ether (150 mL) and methyl iodide (50.4 mL, 808 mmol) was added. The solution was mixed and then allowed to stand. After 4 days, the supernatant was poured off and the precipitate was rinsed with ether and then dried in vacuo. A solution of KOH (1.13 mol) and water (250 mL) was added, and the solution stirred at room temperature until it became homogenous. The solution was then heated to reflux for 2 hours, then cooled to room temperature. 300 mL of water were added and the reaction mixture was poured into a separatory funnel, washed three times with ether, and then acidified with 12N HCl to pH<2. The aqueous phase was extracted three times with ether. These organic phases were combined, washed with 1N HCl and brine, then dried over MgSO₄ and concentrated to afford a yellow oil (18.94 g, 60% yield). No further purification was required. ¹H NMR (400 MHz, CDCl₃) δ: 7.01 (s, 1H), 2.84 (dd, J=13.4, 4.31, 1H) 2.69-2.62 (m, 1H) 2.26 (dd, J=13.4, 1.59, 1H) 1.50-1.39 (m, 2H), 1.31-1.24 (m, 4H), 0.9 (t, J=7.2, 3H). 13C NMR (125 MHz, CDCl₃) δ; 167.6, 153.7, 136.5, 40.1, 34.5, 32.6, 29.9, 22.5, 13.9.

### Example 2.2

### TRANS-3-BUTYLCYCLOBUTANECARBOXYLIC ACID (9)

To a solution of 37% HCl (100 mL), water (67 mL), and THF (166 mL) in a 1 L round-bottom flask at room temperature was added 11 (4 g, 25.9 mmol). The flask was placed in a room temperature water bath and zinc powder (13.47 g, 207.2 mmol) was added in 10 portions at a rate such that the previous portion was almost completely dissolved prior to the next addition (total time was roughly 90 minutes). The reaction was filtered over celite then THF was removed in vacuo. The resulting aqueous phase was extracted three times with ether, the combined organic phases were washed with 1N HCl and brine, then dried over Na₂SO₄ and concentrated. Purification by column chromatography (10% EtOAc in hexanes) afforded 9 as a colorless oil (4.73 g, 59% yield). ¹H NMR (400 MHz, CDCl₃) δ: 3.13-3.06 (m, 1H), 2.42-2.34 (m, 3H), 1.93-1.86 (m, 1H), 1.42 (dt, J=7.7, 7.2, 2H), 1.27 (sextet, J=7.4, 2H) 1.17 (m, 2H), 0.86 (t, J=7.3, 3H). 13C NMR (125 MHz, CDCl₃) δ: 182.0, 36.3, 34.2, 31.3, 28.9, 22.4, 13.9.

### Example 3

### SYNTHESIS OF LEAD INHIBITOR 10

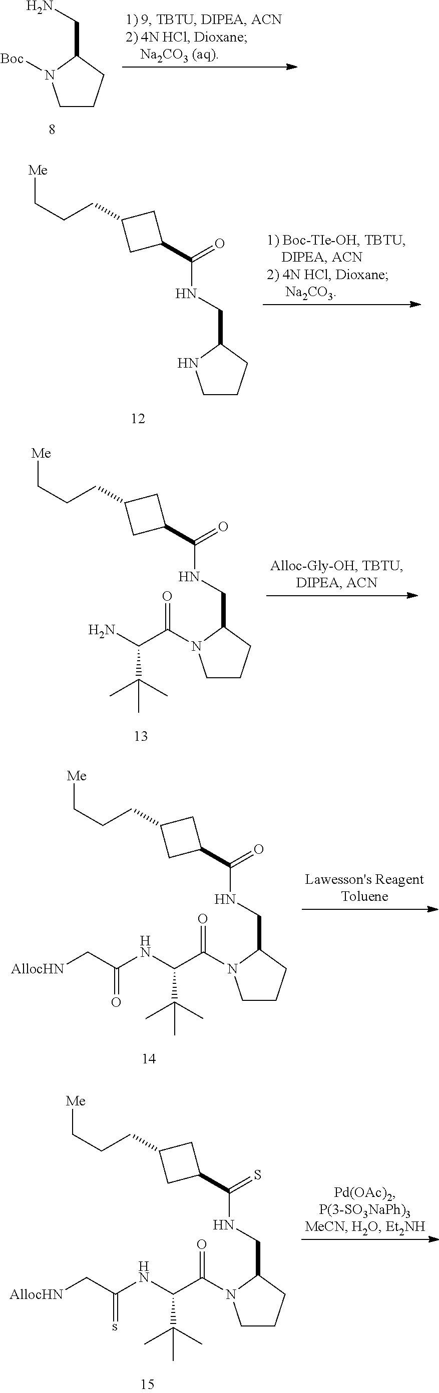

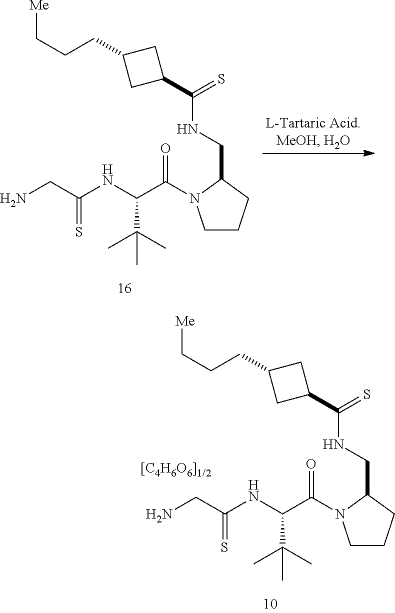 [SMILES: CCCC[C@H]1C[C@H](C(=S)NC[C@H]2CCCN2C(=O)[C@@H](NC(=S)CN)C(C)(C)C)C1 ; CCCC[C@H]1C[C@H](C(=S)NC[C@H]2CCCN2C(=O)[C@@H](NC(=S)CN)C(C)(C)C)C1]

### Example 3.1

### (1s,3R)-3-BUTYL-N((R)-PYRROLIDIN-2-YLMETHYL)CYCLOBUTANECARBOXAMIDETRANS-3-BUTYLCYCLOBUTANECARBOXYLIC ACID (12)

To a solution of 1-Boc-2-(aminomethyl)pyrrolidine (627 mg, 3.13 mmol) in acetonitrile (10 mL) was added 9 (537 mg, 3.45 mmol), DIPEA (819 μL, 4.7 mmol), and TBTU (1.1 g, 3.45 mmol). The mixture was stirred at room temperature for 2 h, then poured into 1N HCl. The aqueous layer was extracted three times with EtOAc, and the combined organic layers were washed with water, saturated NaHCO₃, water, and brine then dried over Na₂SO₄ and concentrated. The residue obtained was dissolved in 4N HCl in dioxane (10 mL), stirred at room temperature for 30 minutes, then poured into 1M aqueous Na₂CO₃. The aqueous phase was extracted three times with ether and the combined organic phases were dried over Na₂SO₄ and concentrated. Purification by column chromatography (0-10% MeOH in CHCl₃) afforded 12 as a white crystalline solid. (314 mg, 33% yield). ¹H NMR (400 MHz, CDCl₃) δ: 5.97 (bs, 1H), 3.4 (ddd, J=13.5, 5.8, 4.6, 1H), 3.25 (ddd, J=11.5, 7.5, 4.6, 1H), 3.04 (ddd, J=13.4, 7.9, 5.2, 1H), 2.97-2.84 (m, 3H), 2.36-2.25 (m, 1H), 2.05 (bs, 1H), 2.89-1.62 (m, 5H) 1.42 (app quartet, J=7.4, 1H), 1.39-1.31 (m, 1H), 1.31-1.14 (m, 4H), 0.86 (t, J=7.3, 3H). ¹³C NMR (125 MHz, CDCl₃) δ: 175.9, 57.8, 46.53, 43.62, 36.7, 36.1, 31.9, 30.51, 30.49, 29.4, 29.1, 25.9, 22.6, 14.1.

### Example 3.2

### (1 S,3R)—N—(((R)-1-((S)-2-AMINO-3,3-DIMETHYLBUTANOYL)PYRROLIDIN-2-YL)METHYL)-3-BUTYLCYCLOBUTANECARBOXAMIDE (13)

To a solution of 12 (1.47 g, 5.36 mmol) in acetonitrile (20 mL) was added S-Boc-tert-Leucine (1.23 g, 5.36 mmol), DIPEA (2.4 mL, 13.4 mmol), HOBt (820 mg, 5.36 mmol), and EDC (1.027 g, 5.36 mmol). The mixture was stirred at room temperature for 6 h, then poured into 1N HCl. The aqueous layer was extracted three times with EtOAc, and the combined organic layers were washed with water, saturated NaHCO₃, water, and brine then dried over Na₂SO₄ and concentrated. The residue obtained was dissolved in 4N HCl in dioxane (25 mL), stirred at room temperature for 30 minutes, then slowly poured into 1M aqueous Na₂CO₃. The aqueous phase was extracted three times with ether and the combined organic phases were dried over Na₂SO₄ and concentrated. Purification by column chromatography (0-10% MeOH in CHCl₃) afforded 13 as a pale yellow oil. (932 mg, 49% yield). ¹H NMR (400 MHz, CDCl₃) δ; 7.29 (bt, J=3.8, 1H), 4.36-4.26 (m, 1H), 3.66-3.53 (m, 2H), 3.45 (ddd, J=13.7, 5.2, 3.9, 1H), 3.29 (s, 1H), 3.24 (ddd J=13.7, 9.3, 4.1, 1H), 3.01-2.87 (m, 1H), 2.38-2.21 (m, 3H), 2.10-1.86 (m, 3H) 1.86-1.73 (m, 3H), 1.63 (bs, 2H), 1.42 (app quartet, J=7.2, 2H), 1.35-1.1 (m, 5H), 0.98 (s, 9H), 0.86 (t, J=7.1, 3H). ¹³C NMR (125 MHz, CDCl₃) δ: 176.3, 175.4, 60.4, 56.8, 47.5, 45.1, 36.7, 36.0, 35.2, 31.9, 30.44, 30.41, 29.4, 29.1, 26.3, 24.0, 22.7, 14.1.

### Example 3.3

### ALLYL(2(((S)-1-((R)-2-(((1 S,3R)-3-BUTYLCYCLOBUTANECARBOXAMIDO)METHYL)PYRROLIDIN-1-YL)-3,3-DIMETHYL-1-OXOBUTAN-2-YL)AMINO)-2-OXOETHYL)CARBAMATE (14)

To a solution of 13 (932 mg, 2.65 mmol) in acetonitrile (10 mL) was added Alloc-Glycine (422.9 mg, 2.66 mmol), DIPEA (713 uL, 3.98 mmol), HOBt (407 mg, 2.66 mmol), and EDC (510, 2.66 mmol). The mixture was stirred at room temperature for 18 h, then poured into 1N HCl. The aqueous layer was extracted three times with EtOAc, and the combined organic layers were washed with water, saturated NaHCO₃, water, and brine then dried over Na₂SO₄ and concentrated to afford 14 as pale yellow oil (1.08 g, 82% yield). The product was used without further purification. ¹H NMR (400 MHz, CDCl₃) δ: 6.80 (bt, J=5.0, 1H), 6.62 (bd J=7.9, 1H), 5.90 (ddt, J=17.2, 10.5, 5.6, 1H), 5.46 (bt, J=4.5, 1H), 5.30 (ddt, J=17.2, 1.3, 1.2, 1H), 5.21 (ddt, J=10.45, 1.3, 1.2, 1H), 4.60-4.56 (m, 1H), 4.19-4.13 (m, 1H), 3.91 (dd J=16.9, 5.7, 1H), 3.84 (dd, J=16.9, 5.7, 1H) 3.72-3.66 (m, 1H), 3.65-3.59 (m, 1H), 3.55 (ddd, 13.8, 5.6, 4.4, 1H), 3.32-3.25 (m, 1H), 2.98-2.91 (m, 1H), 2.35-2.25 (m, 3H), 2.02-1.91 (m, 2H), 1.90-1.75 (m, 4H), 1.46 (app dt J=8.3, 7.8, 2H), 1.27 (sextet, J=7.4, 2H), 1.21-1.14 (m, 2H), 1.00 (s, 9H), 0.87 (t, J=7.3, 3H). 13C NMR (125 MHz, CDCl₃) δ: 176.3, 170.8, 168.9, 156.6, 132.4, 118.2, 66.1, 57.8, 57.4, 48.3, 44.7, 42.8, 36.6, 36.1, 35.2, 31.9, 30.47, 30.39, 29.4, 29.1, 26.46, 24.4, 22.7, 14.1.

### Example 3.4

### ALLYL(2(((S)-1-((R)-2-(((1s,3R)-3-BUTYLCYCLOBUTANECARBOTHIOAMIDO)METHYL)PYRROLIDIN-1-YL)-3,3-DIMETHYL-1-OXOBUTAN-2-YL)AMINO)-2-THIOXOETHYL)CARBAMATE (15)

To a solution of 14 (1.08 g, 2.18 mmol) in toluene (10 mL) was added Lawesson's reagent (880.7 mg, 2.18 mmol). The mixture was stirred at room temperature for 18 h, and then concentrated to an oil. Purification by column chromatography (100% CHCl₃ to 1% MeOH in CHCl₃) afforded 15 (1.024 g, 89%) as a pale yellow oil. ¹H NMR (400 MHz, CDCl₃) δ: 9.22 (bs, 1H), 8.54 (bs, 1H), 5.90 (ddd, J=17.2, 10.5, 5.6, 1H) 5.46 (bt, J=5.3, 1H), 5.31 (ddt, J=17.2, 1.5, 1.4, 1H), 5.23 (ddt, J=10.5, 1.3, 1.2, 1H), 5.16 (bs, J=7.6, 1H), 4.61 (dt, 5.7, 1.3, 1H), 4.43-4.37 (m, 1H), 4.34-4.22 (m, 1H), 4.18 (dd, J=17.11, 6.2, 1H), 4.04-3.97 (m, 1H), 3.92-3.83 (m, 2H), 3.7 (dt, J=10.8, 8.1, 1H), 3.59 (ddd, 14.6, 9.1, 4.0, 1H), 3.38 (pentet, J=8.2, 1H), 2.51-2.43 (m, 1H), 2.41-2.34 (m, 1H), 2.23-2.14 (m, 1H), 2.09-1.84 (m, 6H), 1.47 (q, J=7.6, 2H), 1.33-1.16 (m, 4H), 1.06 (s, 9H), 0.88 (t, J=7.1, 3H). ¹³C NMR (125 MHz, CDCl₃) δ: 209.1, 199.8, 170.6, 157.1, 132.1, 118.4, 66.5, 63.1, 57.1, 52.7, 51.5, 48.3, 45.1, 36.0, 33.11, 33.04, 30.38, 29.68, 29.6, 26.7, 24.0, 22.8, 14.1.

### Example 3.5

### (1s,3R)—N—(((R)-1-((S)-2-(2-AMINOETHANETHIOAMIDO)-3,3-DIMETHYLBUTANOYL)PYRROLIDIN-2-YL)METHYL)-3-BUTYLCYCLOBUTANECARBOTHIOAMIDE (16)

To a solution of 15 (985 mg, 1.88 mmol) in a degassed solution of acetonitrile (3 mL), water (2 mL) and diethylamine (4 mL) was added Triphenylphosphine-3,3′,3″-trisulfonic acid trisodium salt (128.22 mg, 0.224 mmol) and Pd(OAc)₂ (25.1 mg, 0.112 mmol). The mixture was stirred at room temperature for 45 m, then poured into saturated aqueous NaHCO₃. The aqueous phase was extracted three times with EtOAc and the combined organic phases were washed with brine then dried over Na₂SO₄. Purification by column chromatography (100% CHCl₃ to 1% MeOH in CHCl₃) afforded 16 (503.8 mg, 61%) as a white foam. ¹H NMR (400 MHz, CDCl₃) δ: 10.08 (bs, 1H), 9.26 (bs, 1H), 5.14 (s, 1H), 4.45-4.39 (m, 1H), 4.02 (ddd, J=10.8, 7.55, 3.8, 1H), 3.82-3.64 (m, 4H), 3.58 (ddd, 13.9, 9.3, 4.0, 1H), 3.32 (app pentet, J=8.3, 1H), 2.45-2.25 (m, 2H), 2.16-2.06 (m, 1H), 2.03-1.78 (m, 6H), 1.52 (bs, 2H), 1.4 (app quartet, J=7.5, 2H), 1.22 (app pentet, J=7.2, 2H), 1.17-1.1 (m, 2H), 1.04 (s, 9H), 0.81 (t, J=7.23, 3H). ¹³C NMR (125 MHz, CDCl₃) δ: 208.8, 201.9, 170.5, 62.1, 56.8, 52.5, 51.5, 48.4, 45.0, 35.8, 35.6, 32.98, 32.96, 30.3, 29.6, 29.3, 26.8, 24.0, 22.6, 14.2.

### Example 3.6

### (1s,3R)—N—(((R)-1-((S)-2-(2-AMNOETHANETHIOAMIDO)-3,3-DIMETHYLBUTANOYL)PYRROLIDIN-2-YL)METHYL)-3-BUTYLCYCLOBUTANECARBOTHIOAMIDE HEMITARTRATE (10)

To a vial containing 16 (250 mg, 0.56 mmol) and solid tartaric acid (42 mg, 0.284 mmol) was added 1:1 MeOH/water with stirring until the solution became homogenous. The solution was then concentrated in vacuo, then dissolved in ether and concentrated again. The solid thus obtained was used without further purification for biological testing. ¹H NMR (500 MHz, DMSO) δ: 9.65 (bt, J=5.2, 1H), 7.03 (bs, 3H), 5.20 (s, 2H), 4.22 (ddd, J=14.6, 7.6, 1.34, 1H), 3.94 (s, 1H), 3.89 (ddd, J=10.9, 8.5, 2.4, 1H), 3.68 (d, J=2.2, 2H), 3.65-3.52 (m, 3H), 3.45-3.30 (m, 1H), 2.39-2.30 (m, 2H), 2.2-2.07 (m, 2H), 1.98-1.68 (m, 6H), 1.42 (quartet, J=7.4, 2H), 1.24 (pentet, J=7.2, 2H), 1.19-1.12 (m, 2H), 1.00 (s, 9H), 0.84 (t, J=7.3, 3H). ¹³C NMR (125 MHz, DMSO) δ: 208.7, 200.7, 174.6, 168.7, 71.8, 62.7, 56.5, 50.7, 48.0, 46.8, 43.7, 36.2, 35.7, 33.13, 33.09, 30.5, 29.6, 21.9, 26.9, 23.7, 22.6, 14.5.

### II. Biological Examples

### Example 4

### GENERAL ASSAY PREPARATION

In vitro activity was quantified using the octanoylation assay developed by Yang et al. (2008, Proc. Natl. Acad. Sci. USA 105:10750-10755). No pure or soluble form of GOAT that retains enzymatic activity is thus far available. Recombinant poly-His-tagged ghrelin was incubated with radiolabeled octanoyl CoA and membrane fractions isolated from Sf9 cells infected with a GOAT-encoding baculovirus. Recovery of acyl- and des-acyl ghrelin using a Ni²⁺ affinity column was followed by scintillation counting to determine the extent of acyltransferase activity, which was reported as a percentage of control.

GOAT inhibitors were evaluated in vivo using male C57BL/6 mice (n≧3 per group). The small molecules were administered intraperitoneally at 80 mg/kg (formulated in PBS with 1% Tween-80). Quantification of ghrelin species in plasma was performed using commercial ELISA kits. (Taylor, M. S. et al., 2012, Methods in Enzymology 514:205-228.) For in vitro as well as in vivo pharmacokinetic analyses, inhibitor concentration was determined using LC/MS/MS. Activity against GOAT in intact cells was quantified using INS-1 cells stably transfected to express GOAT and preproghrelin. Quantification of ghrelin species was performed by Western immunoblot using antibodies specific to acyl or total ghrelin, as described by Yang et al., 2008 Cell 132:387-396.

### Example 5

### IN VITRO ACTIVITY

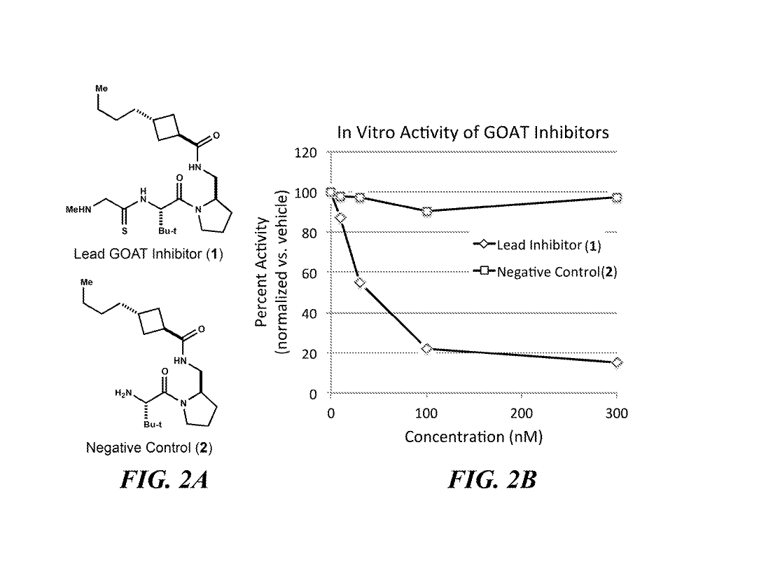

Thiosarcosine derived peptidomimetic Compound 1 (of Example 1.1) and truncated analog Compound 2 (of Example 1.2), which served as a negative control, were screened for GOAT-inhibiting activity using membrane fractions prepared from Sf9 cells infected with baculovirus encoding mouse GOAT as a source of the acyltransferase. The assay detected specific acyl transfer from tritiated octanoyl-CoA to recombinant His-tagged proghrelin. As shown in FIG. 2, Compound 1 inhibited GOAT in vitro in a dose-dependent manner (IC₅₀≈30 nM). Truncated analog Compound 2 was inactive at >10-fold higher concentration.

### Example 6

### TIME DEPENDENCY OF SERUM GHRELIN LEVEL VS. INHIBITOR CONCENTRATION

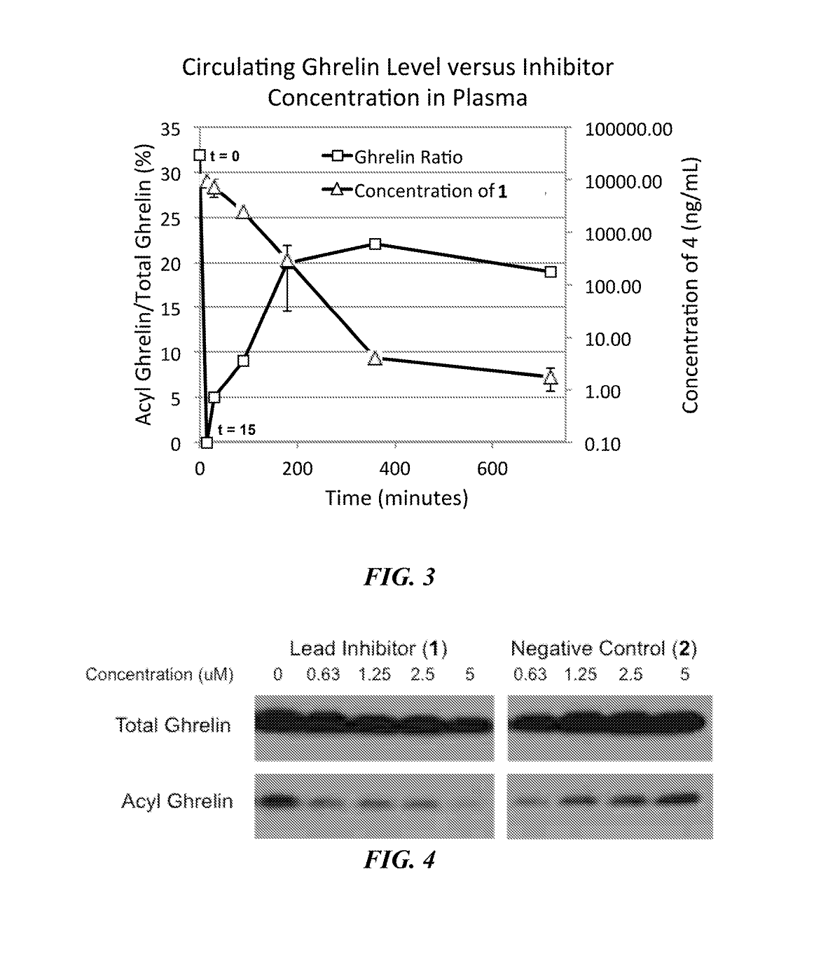

Compound 1 was administered intraperitoneally (IP) to mice (dosed at 80 mg/kg) at time zero and blood samples were collected at indicated timepoints (see FIG. 3). Serum acyl ghrelin: total ghrelin ratios were determined using ELISA with antibodies specific for acyl or des-acyl ghrelin. Inhibitor concentrations were determined using LC/MS/MS detection. As shown in FIG. 3, upon IP administration of Compound 1, acyl-ghrelin in circulation dropped to near undetectable levels within minutes, and recovered after 3 h. Reappearance of acyl-ghrelin tracked with the time-dependent change in inhibitor concentration in plasma.

### Example 7

### PHARMACOKINETIC CHARACTERISTICS IN VITRO

GOAT inhibitor (Compound 1) and control (Compound 2) were added to liver S9 fractions or mouse plasma and the concentrations of each compound over time were quantified using LC/MS/MS. In vitro analyses showed that Compound 1 was stable in mouse plasma and was slowly metabolized by human liver S9 fractions (Table 2).

TABLE 2

| | Plasma | Liver S9 | Measured |
|---|---|---|---|
| | t_{1/2} (min) | t_{1/2} (min) | LogD |
| Lead Inhibitor (1) | >630 | 122 | 1.71 |
| Negative Control (2) | 91 | 125 | N/D |

Molecular parameters for Compound 1 were well within those characteristics of orally bioavailable drugs. In INS-1 cell culture, compounds were administered to cultured INS-1 cells that had been engineered to express GOAT and preproghrelin. After incubating the cells in the presence of the compounds for 18 hours, the cells were lysed and ghrelin species (acyl ghrelin vs. total ghrelin) present in the lysates were quantified by Western immunoblot. As shown in FIG. 4, production of acyl ghrelin was inhibited at concentrations of Compound 1 greater than 1 μM and was substantially completely blocked above 5 μM. Total ghrelin levels were unchanged irrespective of the inhibitor concentration that was present. Control compound 2 was inactive as an inhibitor of acyl ghrelin biosynthesis.

### III. Comparative Results

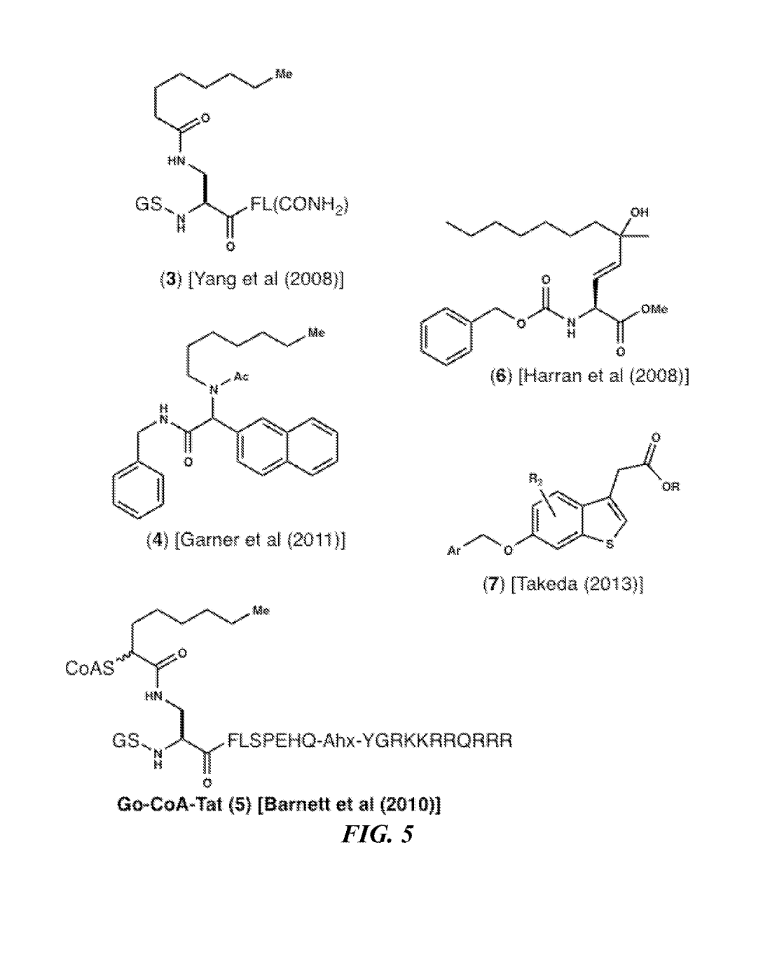

Since the discovery of GOAT, five structures (3, 4, 5, 6, 7) have been reported as inhibitors of the enzyme. See FIG. 5.

It was demonstrated that GOAT was product inhibited and also that an octanoylated pentapeptide derived from the ghrelin N-terminus (3) was effective as a GOAT inhibitor in vitro. Yang, J. et al., 2008, Proc. Natl. Acad. Sci. USA 105:10750-10755. However, lipopeptide (3) was inactive in cell culture.

A 2-napthylglycine derivative (4) was found active against GOAT in an ELISA based assay. Garner, A. et al., 2011, Chem. Commun. 47:7512-7514.

A fusion peptide (5) derived from ghrelin, octanoyl-CoA, and a TAT sequence was also reported to show activity in vitro as well as in vivo. It was further shown to produce compelling phenotypic effects of GOAT inhibition in mice, in particular, a reduction in weight gain for mice fed a high fat diet and improved response to glucose stress in mice treated with (5). Barnett, B. P. et al., 2010, Science 330:1689-1692.

However, no pharmacokinetic data were presented by Barnett et al. for fusion peptide (5) and the observed effects on acyl-ghrelin in vivo were manifested only after 24 h—in contrast to the rapid onset of activity for presently disclosed Compound 1 (cf FIG. 3). Moreover, fusion peptide (5) is a large (MW>3700 Da) fusion peptide harboring a C-terminal TAT sequence for cellular uptake. It lies well outside of structural space typical for orally bioavailable drugs. In contrast, presently disclosed Compound 1 is orally bioavailable (% F=10-15%).

U.S. Pat. No. 8,329,745 discloses a class of vinylglycine-derived inhibitors, such as (6).

Japan patent JP2013/055605 (Takeda Pharmaceuticals) discloses a series of polysubstituted benzothiophenes (7) as GOAT inhibitors. Several inhibitors reported in the Takeda patent (i.e., 7) were reported to inhibit GOAT completely at a concentration of 10 μM in an assay similar to the in vitro GOAT-mediated ghrelin octanoylation assay described herein (Example 4., supra). However, JP2013/055605 disclosed no activity data for the series of polysubstituted benzothiophenes (7) as GOAT inhibitors at lower concentrations, nor were any cellular or in vivo data presented.

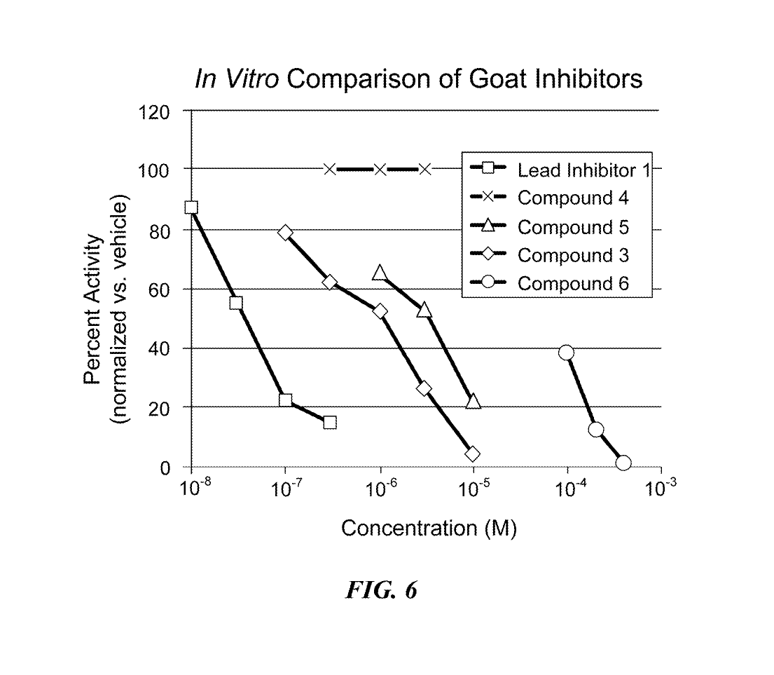

The presently claimed compounds are structurally distinct from any previously reported GOAT inhibitor. Moreover, when compared side-by-side in the ghrelin radio-octanoylation assay performed according to Yang et al. (2008 Proc. Nat. Acad. Sci. USA 105:10750), Compound 1 was by far the most potent inhibitor of GOAT. As shown in FIG. 6, Compound 1 was significantly more potent than the prior art GOAT inhibitors (e.g., compounds 3, 4, 5 and 6).

### REFERENCES

1) National Center for Health Statistics Health E-Stats. Prevalence of overweight, obesity and extreme obesity among adults: United States, trends 1976-80 through 2005-2006. 2008.
2) Flegal, K. M., Carroll, M. D., Ogden, C. L., and Curtin, L. R. 2010. Prevalence and Trends in Obesity Among US Adults, 1999-2008. Journal of the American Medical Association 303:235-241.
3) Finkelstein, E. A., Trogdon, J. G., Cohen, J. W., and Dietz, W. 2009. Annual Medical Spending Attributable To Obesity: Payer- And Service-Specific Estimates. Health Affairs. 28:822-831.
4) Clapham, J. C., Arch, J. R. S., and Tadayyon, M., Pharmacology & Therapeutics. 2001. Anti-obesity drugs: a critical review of current therapies and future opportunities. Pharmacol Ther 89:81-121.
5) Li, M. F., and Cheung, B. M. Y. 2011. Rise and fall of anti-obesity drugs. World J. Diabetes, 2, 19-23
6) Stapleton, J. A. 2009. Trial comes too late as psychiatric side effects end hope for rimonabant. Addiction 104, 277-278.
7) Wren, A. M., Small, C. J., Abbott, C. R., Dhillo W. S., Seal, L. J., Cohen, M. A, Batterham, R L., Taheri, S., Stanley, S. A, Ghatei, M. A, and Bloom, S. R 2001. Ghrelin causes hyperphagia and obesity in rats. Diabetes 50:2540-2547.
8) Wortley, K. E., del Rincon, J.-P., Murray, J. D., Garcia, K., lida, K., Thorner, M. O., and Sleeman, M. W. 2005. Absence of ghrelin protects against early-onset obesity. J. Clin. Invest. 115:3573-3578.
9) Dezaki, K. Kakei, M. and Yada, T. 2007. Ghrelin Uses Gai2 and Activates Voltage-Dependent K+ Channels to Attenuate Glucose-induced Ca2i Signaling and Insulin Release in Islet˜-Cells. Novel Signal Transduction of Ghrelin. Diabetes 56:2319-2327.
10) Kojima, M., Hosoda, H., Date, Y. Nakazato, M., Matsuo, H., and Kangawa, K. 1999. Ghrelin is a growth-hormone-releasing acylated peptide from stomach. Nature 402:656-660.
11) Wren, A. M., Small, C. J., Abbott, C. R., Dhillo W. S., Seal, L. J., Cohen, M. A, Batterham, R L., Taheri, S., Stanley, S. A, Ghatei, M. A, and Bloom, S. R. 2001. Ghrelin causes hyperphagia and obesity in rats. Diabetes 50:2540-2547.
12) Wortley, K. E., del Rincon, J.-P., Murray, J. D., Garcia, K., lida, K., Thomer, M. O., and Sleeman, M. W. 2005. Absence of ghrelin protects against early-onset obesity. J. Clin. Invest. 115:3573-3578.
13) Romero, A, Kirchner, H., Heppner, K., Pfluger, P. T., Tsch6p, M. H., and Nogueiras, R. 2010. GOAT: the master switch for the ghrelin system? European Journal of Endocrinology 163: 1-8.
14) Yang, J., Brown, M. S., Liang, G., Grishin, N. V., and Goldstein, J. L. 2008. Identification of the Acyltransferase that Octanoylates Ghrelin, an Appetite-Stimulating Peptide Hormone. Cell 132:387-396
15) Gutierrez, J. A, Solenberg, P. J., Perkins, D. R., Willency, J. A Knierman, M. D., Jin, Z., Witcher, D. R., Luo, S., Onyia, J. E., and Hale, J. E. 2008. Ghrelin octanoylation mediated by an orphan lipid transferase. Proc. Nat. Acad. Sci. 105:6320:6325.
16) Barnett, B P., Hwang, Y., Taylor, M. S., Kirchner, H., Pfluger, P. T., Bernard, V., Lin, Y. Y., Bowers, E. M., Mukherjee, C., Song, W. J., Longo, P. A, Leahy, D. J., Hussain, M. A., Tsch6p, M. H., Boeke, J. D., and Cole, P. A 2010. Glucose and weight control in mice with a designed ghrelin O-acyltransferase inhibitor. Science 330: 1689-1692
17) Yang, J., Zhao, T.-J., Goldstein, J. L., and Brown, M. S. 2008. Inhibition of ghrelin O-acyltransferase (GOAT) by octanoylated pentapeptides. Proc. Natl. Acad. Sci. USA 105:10750-10755.
18) Taylor, M. S., Hwang, Y., Hsiao, P., Boeke, J. D., and Cole, P. A 2012. Ghrelin O-Acyltransferase Assays and Inhibition. Methods in Enzymology. 514:205-228.
19) Garner, A, and Janda, K. 2011. A small molecule antagonist of ghrelin O-acyltransferase (GOAT). Chem. Commun. 47:7512-7514.
20) Barnett, B. P., Hwang, Y., Taylor, M. S., Kirchner, H., Pfluger, P. T., Bernard, V., Lin, Y. Y., Bowers, E. M., Mukherjee, C., Song, W. J., Longo, P. A, Leahy, D. J., Hussain, M. A., Tsch6p, M. H., Boeke, J. D., and Cole, P. A 2010. Glucose and weight control in mice with a designed ghrelin 0-acyltransferase inhibitor. Science 330: 1689-1692
21) Harran, P. G., Brown, M. S., Goldstein, J. L., Yang, J., and Zhao T. Board of Regents, The University of Texas System (Austin, Tex.), assignee. Ghrelin O-acyltransferase (GOAT) is inhibited with designed small molecules. Methods comprise contacting the GOAT with an inhibitor and detecting a resultant inhibition. U.S. Pat. No. 8,329,745. 2012 Dec. 11.
22) Takakura, N., Banno, Y., Terao, T., Ochida, A, Morimoto, S., Kitamura, S., Tomata, Y., Yasuma, T., Ikoma, M., Masuda, K. Takeda Pharmaceutical Company Limited. Aromatic Ring Compound. Japan patent JP2013/055605. 2013 Aug. 29. SEE ALSO WO2013125732
23) Jin, Z., Tang, B., He, W., and Fu, K., Kaihui. Technology Dev Shanghai Co Ltd., Shanghai Inst. Org. Chem, asignees. Preparation method of (R) or (S)-2-aminomethyl tetrahydropyrrole. China patent CN20091 05529820090724. 2011 Feb. 2.
24) Brannock, K. C., Bell, A, Burpitt, R. D., and Kelly, C. A 1964. Enamine Chemistry. IV. Cycloaddition Reactions of Enamines Derived from Aldehydes and Acyclic Ketones. J. Org. Chem. 29:801-812.
25) Dehmlow, E. V., and Schmidt, S. 1990. Synthesis of Stereoisomeric 3-Substituted Cyclobutanecarboxylic Acid Derivatives. Liebigs Annalen der Chemie. 1990:411-414.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound of Formula (I): 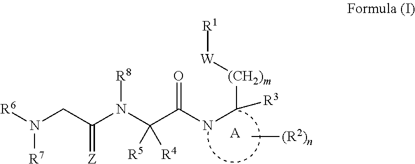
wherein:
A is an N-heterocyclic ring;
W is —C(G)-Y—, —NR⁹—, —O—, —C(G)-, —CH═CH—, or —C≡C—;
G is O or S;
Y is NR⁹ or O;
Z is O, S or NR⁹;
m is 0, 1 or 2;
n is 0, 1, 2, 3 or 4;
R¹ is alkyl, alkenyl, alkynyl, haloalkyl, aryl, aralkyl, heterocyclyl, heterocyclylalkyl, heteroaryl or heteroarylalkyl;
each R² is independently alkyl, aryl, halo, hydroxyl or alkoxy; or
two adjacent R²s together with the atoms to which they are attached form a 5-, 6- or 7-member heterocyclic or carbocyclic ring;
R³ is hydrogen, alkyl, alkenyl or alkynyl;
R⁴ and R⁵ are independently hydrogen, alkyl, aralkyl, alkenyl, alkynyl, aralkenyl, cycloalkyl, cycloalkylalkyl, or haloalkyl; or
R⁴ and R² together with the atoms to which they are attached form an N-heterocyclic ring;
R⁶ and R⁷ are independently hydrogen, alkyl, alkenyl, alkyloxycarbonyl, or alkenyloxycarbonyl; or
R⁶ and R¹ are linked together to form alkylene, alkenylene or alkynylene; or
R⁶ and R⁴ are linked together to form alkylene, alkenylene or alkynylene; or
R⁸ is hydrogen or alkyl; and
R⁹ is hydrogen or alkyl,
a stereoisomer, enantiomer or tautomer thereof, a pharmaceutically acceptable salt thereof, or a prodrug thereof.

2. The compound of claim 1, wherein
W is —C(G)-Y—;
Y is NH or O
G is O or S; and
m is 0, 1 or 2.

3. The compound of claim 2, represented by Formula (II): 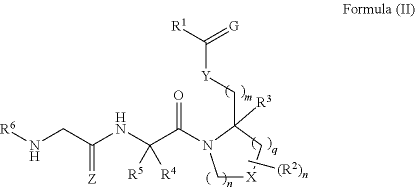
wherein:
p is 0, 1 or 2;
q is 0, 1 or 2;
m is 0, 1 or 2;
n is 0, 1, 2, 3 or 4;
X is —C(R¹⁰)₂—; —O—; —N(R¹⁰)—; or —S—;
G is O or S;
Y is NH or O;
Z is O, S or NR⁹;
R¹ is alkyl, alkenyl, alkynyl, haloalkyl, aryl, aralkyl, heterocyclyl, heterocyclylalkyl, heteroaryl or heteroarylalkyl;
each R² is independently alkyl, aryl, halo, hydroxyl or alkoxy; or
two adjacent R²s together with the atoms to which they are attached form a 5-, 6- or 7-member heterocyclic or carbocyclic ring;
R³ is hydrogen, alkyl, alkenyl or alkynyl;
R⁴ and R⁵ are independently hydrogen, alkyl, aralkyl, alkenyl, alkynyl, aralkenyl, cycloalkyl, cycloalkylalkyl, or haloalkyl;
R⁴ and R² together with the atoms to which they are attached form an N-heterocyclic ring;
R⁶ is hydrogen, alkyl, alkenyl, alkyloxycarbonyl, or alkenyloxycarbonyl; or
R⁶ and R¹ are linked together to form alkylene, alkenylene or alkynylene; or
R⁶ and R⁴ are linked together to form alkylene, alkenylene or alkynylene;
R⁹ is hydrogen or alkyl, and
each R¹⁰ is independently hydrogen, alkyl, halo, hydroxyl or alkoxy; or
R¹⁰ forms a direct bond to an adjacent atom.

4. The compound of claim 3, represented by Formula (IIa), (IIa1), or (IIa2): 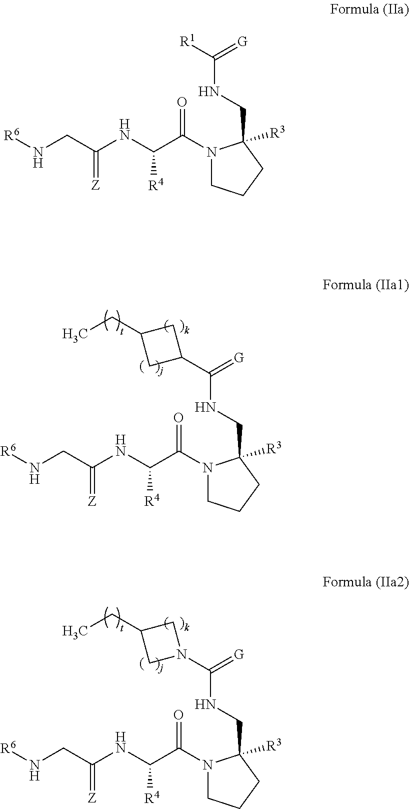
wherein:
k and j are independently 1, 2 or 3;
t is any integer between 0 to 8;
G is O or S;
Z is O, S or NH;
R¹ is alkyl or heterocyclyl;
R³ is hydrogen, alkyl, alkenyl or alkynyl;
R⁴ is alkyl or cycloalkyl; and
R⁶ is hydrogen or alkyl.

5-14. (canceled)

15. The compound of claim 4, wherein the compound is represented by entry 3, 4, 6, 8, 10, 11, 12, 13, 14, 16, 17, 18, 20, 22, 25, 26, or 28 of Table 1.

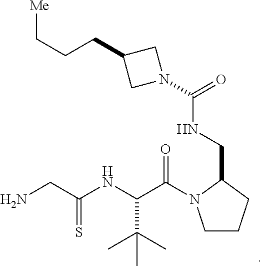

16. The compound of claim 3, wherein:
m is 1;
R¹ is 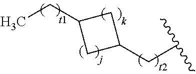
k and j are independently 1, 2 or 3;
t1 and t2 are independently any integer between 0 to 8; and
R⁶ and R⁴ are linked together to form alkylene, alkenylene or alkynylene; two adjacent R²s together with the atoms to which they are attached form a 5-, 6- or 7-member heterocyclic or carbocyclic ring; R⁴ and R² together with the atoms to which they are attached form an N-heterocyclic ring; or R⁶ and R¹ are linked together to form alkylene, alkenylene or alkynylene.

17. The compound of claim 16, wherein the compound is represented by one of the following structural formulas: 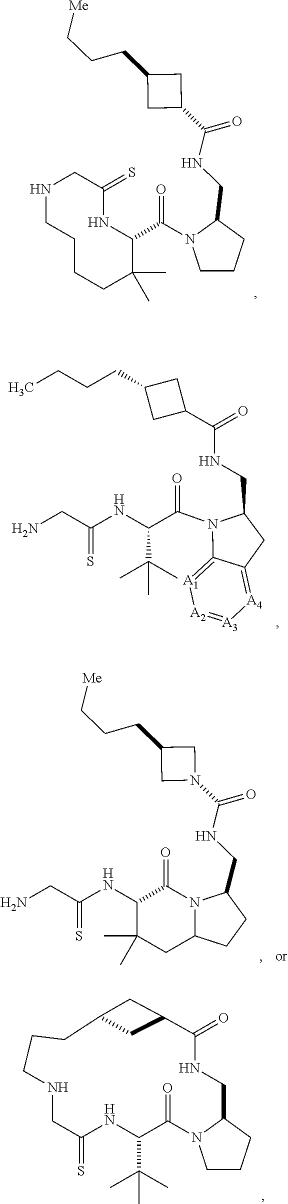
wherein A1, A2, A3 and A4 are independently —CH—; —N— or nothing.

18-23. (canceled)

24. The compound of claim 3, represented by Formula (IIb): 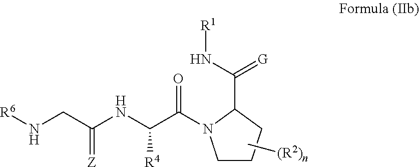
wherein:
n is 0, 1, 2;
G is O or S;
Z is O, S or NH;
R¹ is alkyl or heterocyclyl;
each R² is independently alkyl, aryl, halo, hydroxyl or alkoxy; or
two adjacent R²s together with the atoms to which they are attached form a 5-, 6- or 7-member heterocyclic or carbocyclic ring;
R⁴ is alkyl or cycloalkyl;
R⁶ is hydrogen or alkyl.

25. The compound of claim 24 wherein:
R¹ is 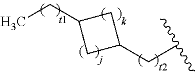
k and j are independently 1, 2 or 3; and
t1 and t2 are independently any integer between 0 to 8.

26. The compound of claim 24, represented by Formula (IIb1) or Formula (IIb2): 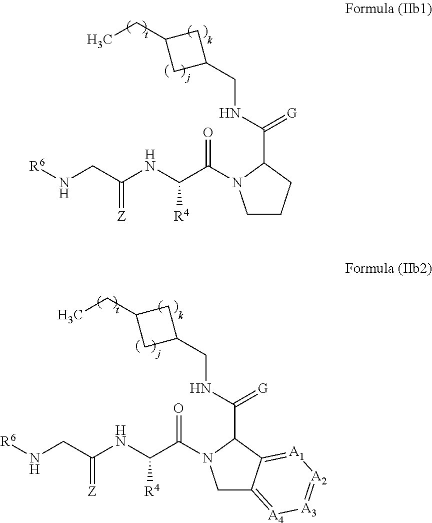
wherein:
k and j are independently 1, 2 or 3;
t is any integer between 0 to 8;
G is O or S;
Z is O, S or NH;
R⁴ is alkyl or cycloalkyl;
R⁶ is hydrogen or alkyl; and
A₁, A₂, A₃ and A₄ is independently —CH—; —N— or nothing.

27-30. (canceled)

31. The compound of claim 26 being:

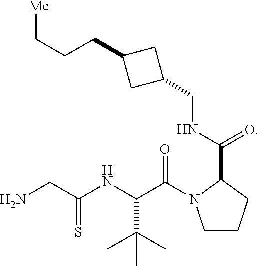 [SMILES: CCCC[C@H]1C[C@H](CNC(=O)[C@H]2CCCN2C(=O)[C@@H](NC(=S)CN)C(C)(C)C)C1]

32. The compound of claim 3, represented by Formula (IIc): 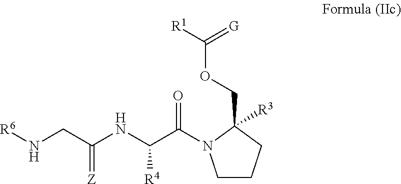
wherein:
G is O or S;
Z is O, S or NH;
R¹ is alkyl or heterocyclyl;
R³ is hydrogen, alkyl, alkenyl or alkynyl;
R⁴ is alkyl or cycloalkyl; and
R⁶ is hydrogen or alkyl.

33. (canceled)

34. The compound of claim 33 being:

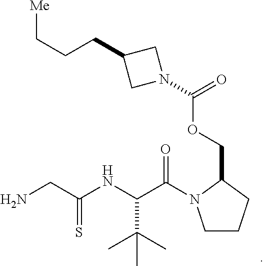

35. The compound of claim 1, wherein:
W is —NR⁹—, —O—, —CH═CH— or —C≡C—;
G is O or S; and
m is 1.

36. (canceled)

37. The compound of claim 35 wherein:
R¹ is 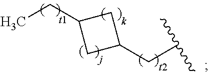
k and j are independently 1, 2 or 3; and
t1 and t2 is independently any integer between 0 to 8.

38. The compound of claim 35 being any one of the following:

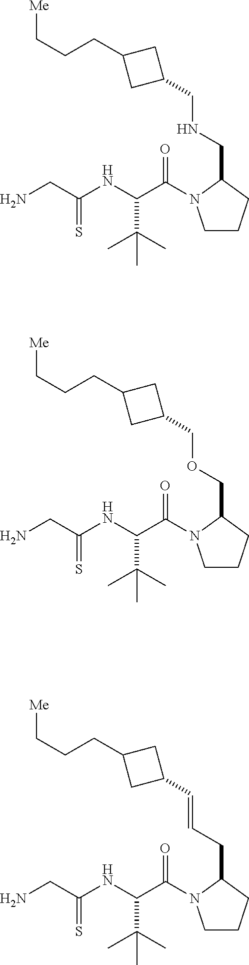

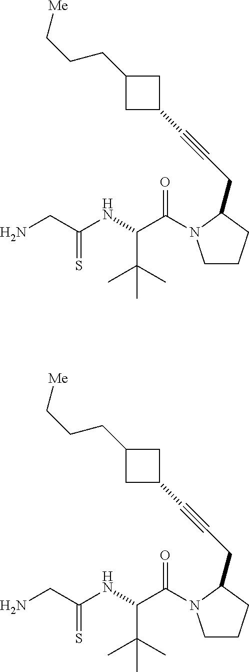

39. (canceled)

40. The compound of claim 39, represented by Formula (III) 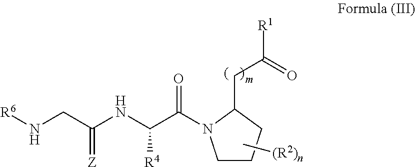
wherein:
n is 0, 1 or 2;
m is 0, 1 or 2;
Z is O, S or NH;
R¹ is alkyl or heterocyclyl;
each R² is independently alkyl, halo, hydroxyl or alkoxy; or
two adjacent R²s together with the atoms to which they are attached form a 5-, 6- or 7-member heterocyclic or carbocyclic ring;
R⁴ is alkyl or cycloalkyl; and
R⁶ is hydrogen or alkyl.

41. The compound of claim 40 being:

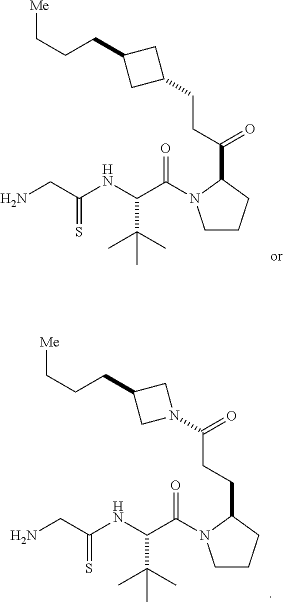

42-45. (canceled)

46. A method for substantially impairing acylation of a ghrelin peptide by a ghrelin O-acyl transferase (GOAT) enzyme, comprising contacting the GOAT enzyme with an effective amount of the compound of claim 1.

47. A method for treating impaired glucose tolerance, insulin resistance, type II diabetes, Prader-Willi syndrome (PWS) or obesity, comprising administering to the subject a therapeutically effective amount of the compound of claim 1.

48-52. (canceled)
